(19) United States
(12) Patent Application Publication
(10) Pub. No.: US 2026/0174653 A1
(43) Pub. Date: Jun. 25, 2026

(54) COMPOSITIONS FOR IMPARTING COLOR AND TONE TO THE HAIR

(71) Applicant: L'Oreal, Paris (FR)

(72) Inventors: Mahima WADHWANI, Moonachie, NJ (US); Emma HOWES, Lake Hiawatha, NJ (US)

(21) Appl. No.: 19/376,692

(22) Filed: Oct. 31, 2025

Related U.S. Application Data
(60) Provisional application No. 63/715,449, filed on Nov. 1, 2024.

(30) Foreign Application Priority Data
Feb. 28, 2025 (FR) 2502028

(51) Int. Cl. A61K 8/41; A61K 8/34; A61K 8/365; A61K 8/44; A61K 8/46; A61Q 5/10

(52) U.S. Cl. CPC A61K 8/411; A61K 8/342; A61K 8/347; A61K 8/365; A61K 8/41; A61K 8/44; A61K 8/466; A61Q 5/10; A61K 2800/4322

(57) ABSTRACT

Disclosed are wash resistant compositions for altering the tone or color of the hair, the compositions comprising a hair coloring agent comprising an oxidation base chosen from toluene-2,5-diamine, acid salts thereof, or solvates thereof, and a coupler system comprising at least one coupler chosen from 1-napthol, 2-amino-4-hydroxyethylaminoanisole sulfate, and derivatives thereof, addition salts thereof, solvates thereof, or solvates of salts thereof; or mixtures thereof, as well as methods of using the compositions.

### CROSS REFERENCE TO RELATED APPLICATION

This application claims priority to U.S. Provisional Application No. 63/715,449, filed Nov. 1, 2024, the contents of which are incorporated herein in their entirety.

### TECHNICAL FIELD

The present disclosure generally relates to compositions and methods for altering the color of hair and for retaining the imparted color and/or tone of the hair.

### BACKGROUND

It is known that consumers desire to use cosmetic and care compositions that enhance the appearance of keratinous substrates such as hair, e.g., by changing the color, style, and/or shape of the hair, and/or by imparting various properties to hair, such as shine and conditioning. Many of the known compositions and processes for enhancing the appearance of the hair involve chemical treatment of the hair.

The process of changing the color of hair, for example, can involve depositing an artificial color onto the hair which provides a different shade or color to the hair, and/or lifting the color of the hair, such as lightening the color of dark hair to lighter shades. The process of coloring the hair with permanent or semi-permanent hair color generally requires the use of hair coloring agents (hair color base compositions) mixed with an oxidative developer composition to form a hair dye mixture.

The degree to which the hair is lightened or darkened with the hair dye mixture corresponds to the degree or level of lightening (lift) and/or color deposit.

A challenge for manufacturers of hair color products is to find new dyes, colorants, or new combinations of dyes or colorants that can either create new shades or color tones on hair, or can achieve the same desired shades or color tones as existing products. However, the choice of such components can pose difficulties insofar as they cannot be detrimental to other attributes such as ease and uniformity of application, rheology or viscosity properties and stability of the compositions, and/or color deposit and target shade formation.

Moreover, color persistence is desired by consumers who use semi-permanent or permanent hair dyes. Hair color often fades with washing, frequently becoming warmer in tone and deviating from the original color. Often, to maintain the desired hair color as it fades, consumers may compensate by applying a direct dye maintenance product such as a direct dye color service or a toning shampoo and/or conditioner which have a small amount of dyes or pigments to compensate for the loss of the permanent oxidative dyes after several washings. However, it would be preferable to minimize color fade of the original dye so that consumers do not need to take multiple additional steps to maintain the desired color.

It would therefore be desirable to provide the consumer with compositions and methods that effectively alter the color of the hair, e.g. lift the color of hair and/or deposit color onto hair in the desired colors and tones, while providing other advantages such as stability of the compositions and shine, conditioning, fiber strength, and/or a healthy appearance to the hair, and also avoiding fast fade out. Balancing these attributes in a single formulation has been a challenging hurdle.

Advantageously, the new dye combinations described herein provide desirable shades without compromising on stability or other formula attributes while exhibiting surprisingly superior color persistence.

### SUMMARY

The disclosure relates to hair color base compositions and hair color toning compositions formed by mixing a hair color base composition with an oxidizing composition. The color and/or tone imparted to the hair with the hair color toning compositions is surprisingly fade-resistant. The disclosure also relates to methods for imparting color and/or tone with improved color persistence to the hair, and to kits comprising hair color bases according to the disclosure.

The hair color base compositions according to the disclosure comprise (i) at least one oxidation base chosen from toluene-2,5-diamine, acid salts thereof, or solvates thereof, and (ii) a coupler system comprising at least one coupler chosen from (ii)(a) 1-napthol, derivatives thereof, addition salts thereof, solvates thereof, or solvates of salts thereof; (ii)(b) 2-amino-4-hydroxyethylaminoanisole sulfate, derivatives thereof, addition salts thereof, solvates thereof, solvates of salts thereof, and (ii)(c) mixtures thereof. This combination of oxidation bases and couplers not only provides surprisingly improved color fastness, but the composition is also surprisingly stable.

The hair color base compositions optionally further comprise (iii) one or more additional components chosen from alkalizing agents, surfactants, fatty compounds, amino acids, carboxylic acids, and additional colorants such as direct dyes, pigments, and additional oxidation dyes. In various embodiments, the alkalizing agents are chosen from mineral and organic alkalizing agents, such as alkanolamines, ammonia, and/or ammonium hydroxide. In various embodiments, the surfactants are chosen from anionic surfactants, nonionic surfactants, and amphoteric surfactants. In various embodiments, the fatty compounds are chosen from lower alkanes, fatty alcohols, esters of fatty acid, esters of fatty alcohol, oils such as mineral, vegetable, animal and synthetic non-silicone oils, non-silicone waxes, and silicones. In various embodiments the amino acids are chosen from amino carboxylic acids and amino sulfonic acids, such as taurine, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In various embodiments the carboxylic acids are chosen from mono-, di-, and tri-carboxylic acids, such as oxalic acid, malonic acid, glutaric acid, succinic acid, adipic acid, glycolic acid, citric acid, tartaric acid, malic acid, sebacic acid, maleic acid, fumaric acid, benzoic acid, citraconic acid, aconitic acid, propane-1,2,3-tricarboxylic acid, and trimesic acid. The hair color base compositions have a pH ranging from about 7 to about 10.5.

Thus, in various embodiments, the hair color base compositions according to the disclosure comprise (i) at least one oxidation base chosen from toluene-2,5-diamine, acid salts thereof, or solvates thereof; (ii) a coupler system comprising at least one coupler chosen from (ii)(a) 1-napthol, derivatives thereof, addition salts thereof, solvates thereof, or solvates of salts thereof; (ii)(b) 2-amino-4-hydroxyethylaminoanisole sulfate, derivatives thereof, addition salts thereof, solvates thereof, solvates of salts thereof, and (ii)(c) mixtures thereof; and (iii) one or more additional components chosen from alkalizing agents, surfactants, fatty compounds, amino acids, carboxylic acids, and additional colorants such as direct dyes, pigments, and additional oxidation dyes, and has a pH ranging from about 7 to about 10.5.

In various embodiments, the hair color base compositions according to the disclosure comprise:
(a) at least one amino acid,
(b) at least one carboxylic acid,
(c) at least one alkalizing agent chosen from organic or mineral alkalizing agents
(d) at least one surfactant mixture comprising at least one amphoteric surfactant and at least one surfactant chosen from anionic and non-ionic surfactants,
(e) at least one fatty compound,
(f) at least one oxidation base chosen from toluene-2,5-diamine, acid salts thereof, or solvates thereof;
(g) a coupler system comprising at least one coupler chosen from:
   (I) 1-napthol, and derivatives thereof, addition salts thereof, solvates thereof, or solvates of salts thereof;
   (II) 2-amino-4-hydroxyethylaminoanisole sulfate, and derivatives thereof, addition salts thereof, solvates thereof, or solvates of salts thereof; and
   (III) mixtures thereof; and
(h) at least one solvent;
   wherein the hair color base composition has a pH ranging from about 7 to about 10.5.

In various embodiments, the hair color base compositions according to the disclosure comprise:
(a) at least one amino acid,
(b) at least one carboxylic acid,
(c) at least one organic alkalizing agent,
(d) at least one surfactant mixture comprising at least one amphoteric surfactant and at least one surfactant chosen from anionic and non-ionic surfactants,
(e) at least one fatty compound,
(f) at least one oxidation base chosen from toluene-2,5-diamine, acid salts thereof, or solvates thereof;
(g) a coupler system comprising at least one coupler chosen from:
   (I) 1-napthol, and derivatives thereof, addition salts thereof, solvates thereof, or solvates of salts thereof;
   (II) 2-amino-4-hydroxyethylaminoanisole sulfate, and derivatives thereof, addition salts thereof, solvates thereof, or solvates of salts thereof; and
   (III) mixtures thereof; and
(h) at least one solvent;
   wherein the hair color base composition has a pH ranging from about 7 to about 10.5.

In various embodiments, the hair color base compositions according to the disclosure comprise:
(a) at least one amino acid,
(b) at least one carboxylic acid,
(c) monoethanolamine,
(d) at least one surfactant mixture comprising at least one amphoteric surfactant and at least one surfactant chosen from anionic and non-ionic surfactants,
(e) at least one fatty compound,
(f) at least one oxidation base chosen from toluene-2,5-diamine, acid salts thereof, or solvates thereof;
(g) a coupler system comprising at least one coupler chosen from:
   (I) 1-napthol, and derivatives thereof, addition salts thereof, solvates thereof, or solvates of salts thereof;
   (II) 2-amino-4-hydroxyethylaminoanisole sulfate, and derivatives thereof, addition salts thereof, solvates thereof, or solvates of salts thereof; and
   (III) mixtures thereof; and
(h) at least one solvent;
   wherein the hair color base composition has a pH ranging from about 7 to about 10.5.

In various embodiments, the hair color base compositions according to the disclosure comprise:
(a) at least one amino acid,
(b) at least one carboxylic acid,
(c) at least one alkalizing agent in addition to monoethanolamine
(d) at least one surfactant mixture comprising at least one amphoteric surfactant and at least one surfactant chosen from anionic and non-ionic surfactants,
(e) at least one fatty compound,
(f) at least one oxidation base chosen from toluene-2,5-diamine, acid salts thereof, or solvates thereof;
(g) a coupler system comprising at least one coupler chosen from:
   (I) 1-napthol, and derivatives thereof, addition salts thereof, solvates thereof, or solvates of salts thereof;
   (II) 2-amino-4-hydroxyethylaminoanisole sulfate, and derivatives thereof, addition salts thereof, solvates thereof, or solvates of salts thereof; and
   (III) mixtures thereof; and
(h) at least one solvent;
   wherein the hair color base composition has a pH ranging from about 7 to about 10.5.

In various embodiments, the hair color base compositions according to the disclosure comprise:
(a) at least one amino acid,
(b) at least one carboxylic acid,
(c) monoethanolamine and ammonia
(d) at least one surfactant mixture comprising at least one amphoteric surfactant and at least one surfactant chosen from anionic and non-ionic surfactants,
(e) at least one fatty compound,
(f) at least one oxidation base chosen from toluene-2,5-diamine, acid salts thereof, or solvates thereof;
(g) a coupler system comprising at least one coupler chosen from:
   (I) 1-napthol, and derivatives thereof, addition salts thereof, solvates thereof, or solvates of salts thereof;
   (II) 2-amino-4-hydroxyethylaminoanisole sulfate, and derivatives thereof, addition salts thereof, solvates thereof, or solvates of salts thereof; and
   (III) mixtures thereof; and
(h) at least one solvent;
   wherein the hair color base composition has a pH ranging from about 7 to about 10.5.

In a further embodiment, the hair color base compositions according to the disclosure comprise (a) at least one amino acid, (b) at least one carboxylic acid, (c) monoethanolamine in an amount ranging from about 0.01% to about 3% by weight, relative to the weight of the hair color base composition, (d) at least one surfactant, (e) at least one fatty compound, (f) at least one oxidation base chosen from toluene-2,5-diamine, acid salts thereof, or solvates thereof, (g) a coupler system comprising at least one coupler chosen from (I) 1-napthol, and derivatives thereof, addition salts thereof, solvates thereof, or solvates of salts thereof; (II) 2-amino-4-hydroxyethylaminoanisole sulfate, and derivatives thereof, addition salts thereof, solvates thereof, or solvates of salts thereof; and (III) mixtures thereof, and (h) at least one solvent, wherein the hair color base composition has a pH ranging from about 7 to about 10.5.

In yet a further embodiment, the hair color base compositions according to the disclosure comprise (a) from about 0.1% to about 2% taurine by weight, relative to the weight of the hair color base composition, (b) from about 0.1% to about 3% citric acid by weight, relative to the weight of the hair color base composition, (c) from about 0.1% to about 3% monoethanolamine by weight, relative to the weight of the hair color base composition, (d) at least one surfactant, (e) at least one fatty compound chosen from C12-C22 fatty alcohols, (f) at least one oxidation base chosen from toluene-2,5-diamine, acid salts thereof, or solvates thereof, (g) a coupler system comprising at least one coupler chosen from (I) 1-napthol, and derivatives thereof, addition salts thereof, solvates thereof, or solvates of salts thereof; (II) 2-amino-4-hydroxyethylaminoanisole sulfate, and derivatives thereof, addition salts thereof, solvates thereof, or solvates of salts thereof; and (III) mixtures thereof, and (h) at least one solvent, wherein the hair color base composition has a pH ranging from about 7 to about 10.5.

The disclosure also relates to methods for altering the tone or color of the hair, the methods comprising applying to the hair a hair color toning composition formed by mixing an oxidizing composition with a hair color base composition according to the disclosure.

In yet further embodiments, the disclosure relates to kits comprising a hair color base according to the disclosure.

### BRIEF DESCRIPTION OF THE FIGURES

FIGS. 1A-1B show a comparison of hair treated with a hair color toning composition according to the disclosure (I1) and processed for 5 minutes or 10 minutes after one wash and after ten washes (1A), and hair treated with a hair color toning composition not according to the disclosure (C1) and processed for 5 minutes or 10 minutes after one wash and after ten washes (1B). As can be seen, the hair treated with I1 and C1 at T₁ appeared to be very close in color and tone, whereas the hair treated with I1 retained more color at T₁₀ than hair treated with C1.

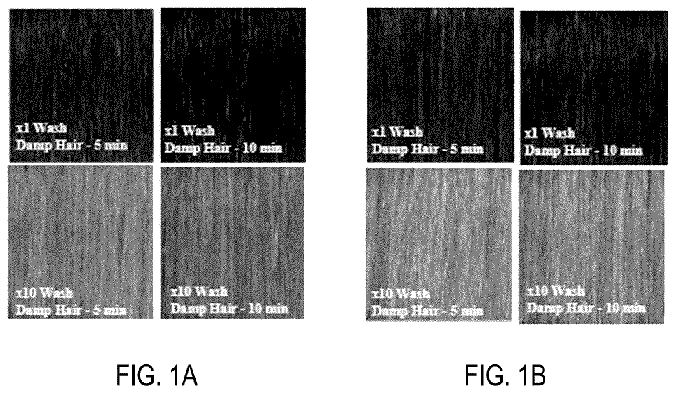

FIGS. 2A-2B show a comparison of hair treated with a hair color toning composition according to the disclosure (I3) and processed for 10 minutes after one wash and ten washes (FIG. 2A), and hair treated with a hair color toning composition not according to the disclosure (C4) and processed for 20 minutes after one wash and ten washes (FIG. 2B). As can be seen, the hair treated with I3 and C4 at T₁ appeared to be very close in color and tone, whereas the hair treated with I3 retained more color at T₁₀ than hair treated with C4.

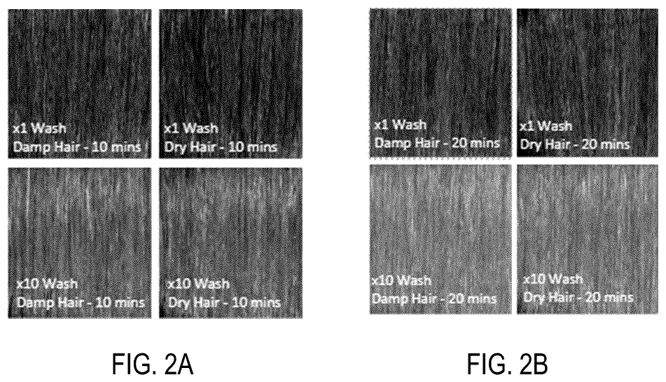

FIGS. 3A-3B show a comparison of hair treated with a hair color toning composition according to the disclosure (I5) and processed for 10 minutes after one wash, ten washes, twenty washes, and twenty-eight washes (FIG. 3A), and hair treated with a hair color toning composition not according to the disclosure (C5) and processed for 20 minutes after one wash, ten washes, twenty washes, and twenty-eight washes (FIG. 3B). As can be seen, the hair treated with I5 and C5 at T₁ appeared to be very close in color and tone, whereas the hair treated with I5 retained more color at T₁₀, T₂₀, and T₂₈ than hair treated with C5.

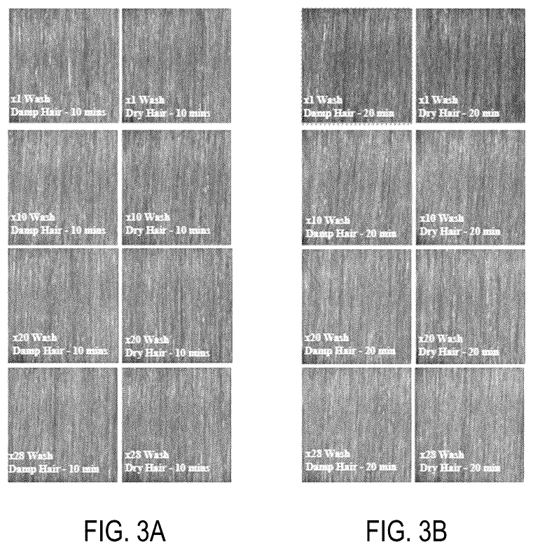

FIGS. 4A-4B show a comparison of hair treated with a hair color toning composition according to the disclosure (I7) and processed for 10 minutes after one wash, ten washes, twenty washes, and twenty-eight washes (FIG. 4A), and hair treated with a hair color toning composition not according to the disclosure (C7) and processed for 20 minutes after one wash, ten washes, twenty washes, and twenty-eight washes (FIG. 4B). As can be seen, the hair treated with I7 and C7 at T₁ appeared to be very close in color and tone, whereas the hair treated with I7 retained more color at T₁₀, T₂₀, and T₂₈ than hair treated with C7.

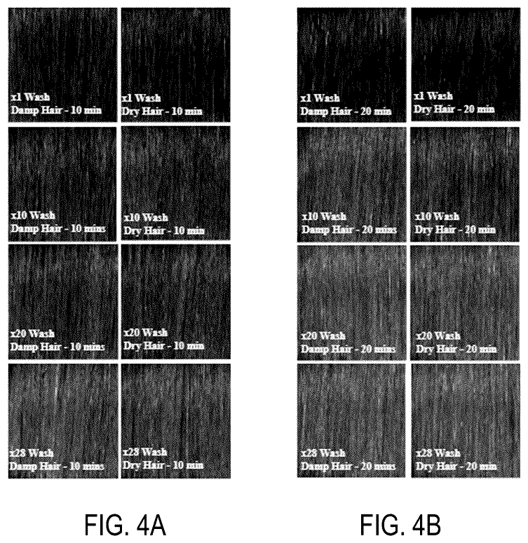

### DETAILED DESCRIPTION

The disclosure relates to compositions for altering the color or tone of the hair. The hair color base and hair color toning compositions comprise a hair coloring agent comprising at least one oxidation base chosen from toluene-2,5-diamine, and a coupler system comprising at least one coupler chosen from 1-napthol and/or 2-amino-4-hydroxyethylaminoanisole sulfate. The hair color toning compositions are prepared by mixing a hair color base according to the disclosure with an oxidizing agent. Surprisingly, the hair color toning compositions have improved color persistence compared to compositions that do not comprise the combination of oxidation base and coupler. The disclosure further relates to methods of using the hair color base and hair color toning compositions, and kits comprising the hair color base compositions.

### I. Hair Color Base and Hair Color Toning Compositions

Hair color base and hair color toning compositions according to the disclosure comprise at least one hair coloring agent comprising an oxidation base chosen from toluene-2,5-diamine, and a coupler system comprising at least one coupler chosen from 1-napthol and/or 2-amino-4-hydroxyethylaminoanisole sulfate. The compositions further comprise at least one additional component chosen from solvents, surfactants, fatty compounds, amino acids, carboxylic acids, alkalizers, and/or additional hair coloring agents/components. In some embodiments, the hair color base and hair color toning compositions are free or substantially free of oxidizing agents.

### Hair Coloring Agent

The hair color base and hair color toning compositions according to the disclosure comprise a hair coloring agent comprising (i) toluene-2,5-diamine as an oxidation base, and (ii) at least one coupler chosen from (a) 1-napthol, and derivatives thereof, addition salts thereof, solvates thereof, or solvates of salts thereof, (b) 2-amino-4-hydroxyethylaminoanisole sulfate, and derivatives thereof, addition salts thereof, solvates thereof, or solvates of salts thereof, and (c) mixtures thereof.

It should be understood that reference herein to toluene-2,5-diamine, 1-napthol, and 2-amino-4-hydroxyethylaminoanisole specifically includes derivatives, salts, and solvates thereof unless otherwise specified. Non-limiting examples of useful salts include toluene-2,5-diamine sulfate, 1-naphthalenol, potassium salt, 2-amino-4-hydroxyethylaminoanisole sulfate. For example, in various embodiments, 2-amino-4-hydroxyethylaminoanisole is used as the sulfate salt.

### Additional Hair Coloring Agent(s)

It will be possible to provide additional shades and tones by including one or more additional hair coloring agents in the hair color base or hair color toning composition without significantly impacting color persistence properties. Therefore, in various embodiments, the hair color base and/or hair color toning compositions according to the disclosure may comprise one or more additional hair coloring agent chosen from oxidative dyes (one or more oxidation bases optionally combined with one or more couplers), direct dyes, and pigments. It should be understood that reference herein to additional oxidation dyes specifically includes derivatives, salts, and solvates thereof unless otherwise specified. Non-limiting examples of useful salts include toluene-2,5-diamine sulfate, 1-naphthalenol, potassium salt, 2-amino-4-hydroxyethylaminoanisole sulfate.

By way of example, the additional oxidation bases (other than toluene-2,5-diamine), may be chosen from para-phenylenediamines, bis(phenyl)alkylenediamines, para-aminophenols, ortho-aminophenols and heterocyclic bases, and the addition salts thereof.

Among the para-phenylenediamines that may be mentioned, for example, are para-phenylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-methoxymethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the addition salts thereof with an acid.

Among the para-phenylenediamines mentioned above, para-phenylenediamine, para-toluenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the addition salts thereof with an acid, are particularly preferred.

Among the bis(phenyl)alkylenediamines that may be mentioned, for example, are N,N′-bis(p-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(p-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the addition salts thereof.

Among the para-aminophenols that may be mentioned, for example, are para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the addition salts thereof with an acid.

Among the ortho-aminophenols that may be mentioned, for example, are 2-aminophenol, 2-amino-5-methylphenol, 2-amino-6-methylphenol and 5-acetamido-2-aminophenol, and the addition salts thereof.

Among the heterocyclic bases that may be mentioned, for example, are pyridine derivatives, pyrimidine derivatives and pyrazole derivatives.

Among the pyridine derivatives that may be mentioned are the compounds described, for example, in patents GB 1 026 978 and GB 1 153 196, for instance 2,5-diaminopyridine, 2-(4-methoxyphenyl)amino-3-aminopyridine and 3,4-diaminopyridine, and the addition salts thereof.

Other pyridine oxidation bases can include the 3-aminopyrazolo[1,5-a]pyridine oxidation bases or the addition salts thereof described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-morpholin-4-ylpyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-morpholin-4-ylpyrazolo[1,5-a]pyrid-3-ylamine, pyrazolo[1,5-a]pyridine-3,5-diamine, 5-morpholin-4-ylpyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol, 2-β-hydroxyethoxy-3-amino-pyrazolo[1,5-a]pyridine; 2-(4-diméthylpyperazinium-1-yl)-3-amino-pyrazolo[1,5-a]pyridine; and the addition salts thereof.

More particularly, oxidation bases can be 3-aminopyrazolo-[1,5-a]-pyridines, preferably substituted on carbon atom 2 by (a) one (di)(C₁-C₆)(alkyl)amino group wherein said alkyl group can be substituted by at least one hydroxy, amino, imidazolium group, (b) one heterocycloalkyl group containing from 5 to 7 members chain, and from 1 to 3 heteroatomes, potentially cationic, potentially substituted by one or more (C1-C6)alkyl, such as di(C1-C4)alkylpiperazinium, or (c) one (C1-C6)alkoxy potentially substituted by one or more hydroxy groups such as α-hydroxyalkoxy, and the addition salts thereof.

Among the pyrimidine derivatives that may be mentioned are the compounds described, for example, in the patents DE 2359399; JP 88-169571; JP 05-63124; EP 0770375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and their addition salts and their tautomeric forms, when a tautomeric equilibrium exists.

Among the pyrazole derivatives that may be mentioned are the compounds described in the patents DE 3843892, DE 4133957 and patent applications WO 94/08969, WO 94/08970, FR-A-2 733 749 and DE 195 43 988, such as 4,5-diamino-1-methyl-pyrazole, 4,5-diamino-1-(p-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(p-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropyl-pyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(p-hydroxyethyl)amino-1-methylpyrazole, and the addition salts thereof. 4,5-Diamino-1-(p-methoxyethyl)pyrazole may also be used. A 4,5-diaminopyrazole will preferably be used, and even more preferentially 4,5-diamino-1-(β-hydroxyethyl)pyrazole and/or a salt thereof.

Pyrazole derivatives that may also be mentioned include diamino-N,N-dihydro-pyrazolopyrazolones and especially those described in patent application FR-A-2 886 136, such as the following compounds and the addition salts thereof: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-di-(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one, 2,3-diamino-6-hydroxy-6,7-dihydro-1H,5H-pyrazolo[1,2-a]pyrazol-1-one.

4,5-Diamino-1-(β-hydroxyethyl)pyrazole and/or 2,3-diamino-6,7-dihydro-1H,5H-pyrazolo[1,2-a]pyrazol-1-one and/or a salt thereof will preferentially be used as heterocyclic bases.

Compositions and/or systems according to embodiments of the disclosure may optionally further comprise additional couplers advantageously chosen from those conventionally used in the dyeing or coloring of keratinous substrates. These are in addition to 1-napthol and/or 2-amino-4-hydroxyethylaminoanisole sulfate and their derivatives thereof, their addition salts thereof, their solvates thereof, or their solvates of salts thereof.

Among these additional couplers, mention may be made especially of meta-phenylenediamines, meta-aminophenols, meta-diphenols, naphthalene-based couplers and heterocyclic couplers, and also the addition salts thereof.

Mention may be made, for example, of 2-methyl-5-aminophenol, 5-N-(ß-hydroxyethyl)amino-2-methylphenol, 3-aminophenol, 5-amino-6-chloro-o-cresol (3-amino-2-chloro-6-methylphenol), 1,3-dihydroxybenzene, 1,3-dihydroxy-2-methylbenzene, 4-chloro-1,3-dihydroxybenzene, 2,4-diamino-1-(ß-hydroxyethyloxy)benzene, 2-amino-4-(ß-hydroxyethylamino)-1-methoxybenzene, 1,3-diaminobenzene, 1,3-bis(2,4-diamino-phenoxy)propane, 3-ureidoaniline, 3-ureido-1-dimethylaminobenzene, sesamol, 1-ß-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(ß-hydroxyethyl)-amino-3,4-methylenedioxybenzene, 2,6-bis(ß-hydroxyethylamino)toluene, 6-hydroxy-indoline, 2,6-dihydroxy-4-methylpyridine, 1-H-3-methylpyrazol-5-one, 1-phenyl-3-methyl-pyrazol-5-one, 2,6-dimethylpyrazolo[1,5-b]-1,2,4-triazole, 2,6-dimethyl[3,2-c]-1,2,4-triazole and 6-methylpyrazolo[1,5-a]benzimidazole, the addition salts thereof with an acid, and mixtures thereof.

In general, the addition salts of the oxidation bases and couplers that may be used include addition salts with an acid such as the hydrochlorides, hydrobromides, sulfates, citrates, succinates, tartrates, lactates, tosylates, benzenesulfonates, phosphates and acetates.

The oxidation base(s) may be present in an amount ranging from about 0.001% to 10% by weight, such as from about 0.001% to 6% by weight, relative to the total weight of the hair color base or hair color toning composition.

The coupler(s), if they are present, may be present in an amount ranging from about 0.001% to 10% by weight, such as from about 0.001% to 6% by weight, relative to the total weight of the hair color base or hair color toning composition.

### Direct Dyes

Hair color base and hair color toning compositions according to various embodiments of the disclosure may optionally comprise one or more synthetic or natural direct dyes, for example chosen from anionic and nonionic species, preferably cationic or nonionic species, either as sole dyes or in addition to the oxidation dye(s).

Examples of suitable direct dyes that may be mentioned include azo direct dyes; (poly)methine dyes such as cyanins, hemicyanins and styryls; carbonyl dyes; azine dyes; nitro(hetero)aryl dyes; tri(hetero)arylmethane dyes; porphyrin dyes; phthalocyanin dyes, and natural direct dyes, alone or as mixtures.

Preferably, direct dyes are cationic direct dyes. Mention may be made of the hydrazono cationic dyes of formulas (Va) and (V′a), the azo cationic dyes (Via) and (VI′a) and the diazo cationic dyes (Vila) below:

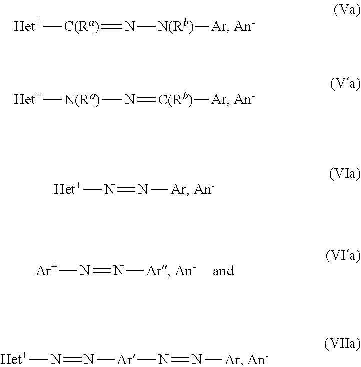

in which formulas (Va), (V′a), (Via), (VI′a) and (Vila):
Het⁺ represents a cationic heteroaryl radical, preferably bearing an endocyclic cationic charge, such as imidazolium, indolium or pyridinium, optionally substituted preferentially with one or more (C₁-C₈) alkyl groups such as methyl;
Ar⁺ representing an aryl radical, such as phenyl or naphthyl, bearing an exocyclic cationic charge, preferentially ammonium, particularly tri(C₁-C₈)alkylammonium such as trimethylammonium;
Ar represents an aryl group, especially phenyl, which is optionally substituted, preferentially with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or alternatively Ar represents a julolidine group;
Ar′ is an optionally substituted divalent (hetero)arylene group such as phenylene, particularly para-phenylene, or naphthalene, which are optionally substituted, preferentially with one or more groups (C₁-C₈)alkyl, hydroxyl or (C₁-C₈)alkoxy;
Ar″ is an optionally substituted (hetero)aryl group such as phenyl or pyrazolyl, which are optionally substituted, preferentially with one or more groups (C₁-C₈)alkyl, hydroxyl, (di)(C₁-C₈)(alkyl)amino, (C₁-C₈)alkoxy or phenyl;
R^{a} and R^{b}, which may be identical or different, represent a hydrogen atom or a group (C₁-C₈)alkyl, which is optionally substituted, preferentially with a hydroxyl group; or alternatively the substituent R^{a} with a substituent of Het⁺ and/or R^{b} with a substituent of Ar and/or R^{a} with R^{b} form, together with the atoms that bear them, a (hetero)cycloalkyl; particularly, R^{a} and R^{b} represent a hydrogen atom or a group (C₁-C₄)alkyl, which is optionally substituted with a hydroxyl group;
An⁻ represents an anionic counter-ion such as mesylate or halide.

In particular, mention may be made of the azo and hydrazono cationic dyes bearing an endocyclic cationic charge of formulae (Va), (V′a) and (Via) as defined previously. More particularly those of formulae (Va), (V′a) and (Via) derived from the dyes described in patent applications WO 95/15144, WO 95/01772 and EP-714954.

Preferentially, the cationic part is derived from the following derivatives (Va-1) and (Via-1):

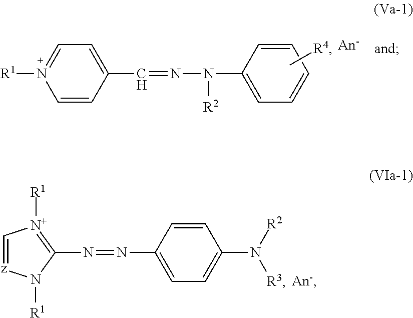

with:
   R¹ representing a (C₁-C₄) alkyl group such as methyl;
   R² and R³, which are identical or different, represent a hydrogen atom or a (C₁-C₄)alkyl group, such as methyl; and
   R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; particularly, R⁴ is a hydrogen atom,
Z represents a CH group or a nitrogen atom, preferentially CH; and
An⁻ represents an anionic counter-ion such as mesylate or halide.

The dye of formulae (Va-1) and (Via-1) can be chosen from Basic Red 51, Basic Yellow 87 and Basic Orange 31 or derivatives thereof:

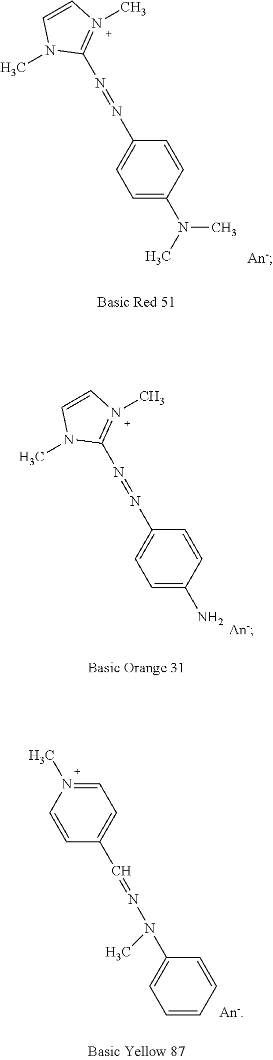

Among the natural direct dyes, mention may be made of lawsone, juglone, alizarin, purpurin, carminic acid, kermesic acid, purpurogallin, protocatechaldehyde, indigo, isatin, curcumin, spinulosin, apigenidin and orceins. Extracts or decoctions containing these natural dyes and in particular henna-based poultices or extracts may also be used.

When they are present, the one or more direct dyes more represent from about 0.001% to 10% by weight, such as from about 0.005% to 5% by weight, of the total weight of the hair color base or hair color toning composition.

### Amino Acid(s)

The hair color base and/or hair color toning compositions according to the disclosure optionally comprise at least one amino acid. As used herein, the term “amino acid” includes amino carboxylic acids such as proteinogenic amino acids, amino sulfonic acids, and salts thereof. It should be understood that salts of amino acids are included whether or not specified, unless expressly stated otherwise.

Amino carboxylic acids are simple organic compounds containing both a carboxylic acid group (—OOH) and an amino group (—NH₂). Amino sulfonic acids are simple organic compounds containing both a sulfonic acid group (—SO₂OH) and an amino group (—NH₂). Accordingly, the amino carboxylic acids useful according to the disclosure may, in certain embodiments, be chosen from compounds of Formula (I) and Formula (II):

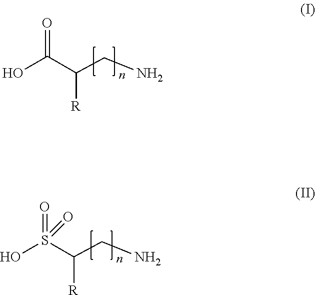

wherein: R represents a hydrogen atom, a linear or branched, preferably linear, C₁-C₅ alkyl group, said alkyl group being optionally substituted with at least one group chosen from hydroxyl, —C(O)—OH, —S(O)₂—OH, —C(O)—O— and M⁺, and S(O)₂—O⁻ and M⁺, with M⁺ representing a cationic counter-ion such as an alkali metal, alkaline earth metal, or ammonium, and n is 0 or 1.

The amino acids may be in their non-ionized form (I) and (II) or in their ionized or betaine form (I′) and (II′):

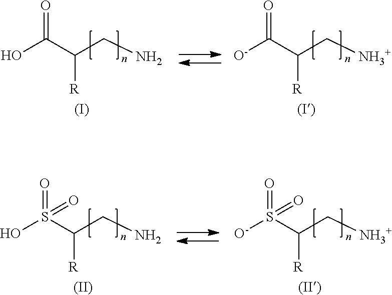

wherein R and n are as defined above.

The one or more amino acids may also be in their conjugate base form (Ib) and (IIb):

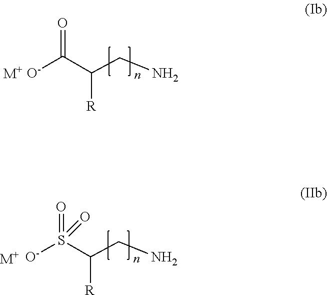

wherein R and n are as defined above.

Well-known amino acids include the twenty amino acids that form the proteins of living organisms (standard proteinogenic amino acids): alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. The amino carboxylic acids of the instant disclosure, however, are not limited to the standard proteinogenic amino acids.

Non-limiting examples of amino sulfonic acids include aminomethane sulfonic acid, 2-aminoethane sulfonic acid (taurine), aminopropane sulfonic acid, aminobutane sulfonic acid, aminohexane sulfonic acid, aminoisopropyl sulfonic acid, aminododecyl sulfonic acid, aminobenzene sulfonic acid, aminotoulene sulfonic acid, sulfanilic acid, chlorosulfanilic acid, diamino benzene sulfonic acid, amino phenol sulfonic acid, amino propyl benzene sulfonic acid, amino hexyl benzene sulfonic acid, and mixtures of two or more thereof.

In some cases, charged amino acids may be used. Non-limiting examples of charged amino acids include arginine, lysine, aspartic acid, and glutamic acid. In some cases, polar amino acids are useful. Non-limiting examples of polar amino acids include glutamine, asparagine, histidine, serine, threonine, tyrosine, cysteine, methionine, and tryptophan.

In some cases, hydrophobic amino acids may be employed. Non-limiting examples of hydrophobic amino acids include alanine, isoleucine, leucine, phenylalanine, valine, proline, and glycine.

In certain exemplary embodiments, compositions according to the disclosure include at least one amino acid chosen from glycine, alanine, serine, beta-alanine, taurine, sodium glycinate, sodium alaninate, sodium serinate, lithium beta-alanine, sodium taurate, or combinations of two or more thereof.

In further exemplary embodiments, compositions according to the disclosure include amino acids chosen from aspartic acid, cysteine, glycine, lysine, methionine, proline, tyrosine, phenylalanine, carnitine, taurine, or a salt thereof.

In one exemplary embodiment, the compositions include at least taurine. In a further embodiment, the only amino acid in the composition is taurine. In a further embodiment, the compositions include at least glycine. In yet a further embodiment, the only amino acid in the composition is glycine. In various embodiments, the compositions include both taurine and glycine.

The total amount of the at least one amino acid may range from about 0.01% to about 10% by weight, relative to the total weight of the hair color base or hair color toning composition. For example, in some embodiments, the total amount of the at least one amino acid may range from about 0.05% to about 5%, such as about 0.1% to about 5%, about 0.1% to about 4%, about 0.1% to about 3%, about 0.1% to about 2.5%, about 0.1% to about 2.4%, about 0.1% to about 2.3%, about 0.1% to about 2.2%, about 0.1% to about 2.1%, about 0.1% to about 2%, about 0.1% to about 1.9%, about 0.1% to about 1.8%, about 0.1% to about 1.7%, about 0.1% to about 1.6%, about 0.1% to about 1.5%, about 0.1% to about 1.4%, about 0.1% to about 1.3%, about 0.1% to about 1.2%, about 0.1% to about 1.1%, about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, or about 0.1% to about 0.3% by weight, relative to the total weight of the hair color base or hair color toning composition. In other embodiments, the total amount of the at least one amino acid ranges from about 0.5% to about 5%, about 0.5% to about 4%, about 0.5% to about 3%, about 0.5% to about 2.5%, about 0.5% to about 2.4%, about 0.5% to about 2.3%, about 0.5% to about 2.2%, about 0.5% to about 2.1%, about 0.5% to about 2%, about 0.5% to about 1.9%, about 0.5% to about 1.8%, about 0.5% to about 1.7%, about 0.5% to about 1.6%, about 0.5% to about 1.5%, about 0.5% to about 1.4%, about 0.5% to about 1.3%, about 0.5% to about 1.2%, about 0.5% to about 1.1%, about 0.5% to about 1%, about 0.5% to about 0.9%, about 0.5% to about 0.8%, about 0.5% to about 0.7%, or about 0.5% to about 0.6% by weight, relative to the total weight of the hair color base or hair color toning composition.

The total amount of the at least one amino acid may, in certain embodiments, be about 0.05%, about 0.06%, about 0.07%, about 0.08%, about 0.09%, about 0.1%, about 0.11%, about 0.12%, about 0.13%, about 0.14%, about 0.15%, about 0.16%, about 0.17%, about 0.18%, about 0.19%, about 0.2%, about 0.21%, about 0.22%, about 0.23%, about 0.24%, about 0.25%, about 0.26%, about 0.27%, about 0.28%, about 0.29%, about 0.3%, about 0.31%, about 0.32%, about 0.33%, about 0.34%, or about 0.35% by weight, relative to the total weight of the hair color base or hair color toning composition. In yet further embodiments, the total amount of the amino acid may be about 0.4%, about 0.5%, about 0.6%, about 0.7%, about 0.8%, about 0.9%, about 1%, about 1.1% about 1.2%, about 1.3%, about 1.4%, about 1.5%, about 1.6%, about 1.7% about 1.8%, about 1.9%, or about 2% by weight, relative to the total weight of the hair color base or hair color toning composition. It is to be understood that any of the above-recited numbers may provide an upper or lower boundary for a range of the total amount of the at least one amino acid.

### Carboxylic Acid(s)

The hair color base and/or hair color toning compositions according to the disclosure optionally include at least one carboxylic acid. As used herein, the term “carboxylic acid” includes salts of carboxylic acids whether or not specified, unless expressly stated otherwise. In certain embodiments, the carboxylic acids include non-polymeric mono, di, and/or tricarboxylic acid which are organic compounds having one (mono), two (di), or three (tri) carboxylic acid groups (—COOH). The non-polymeric mono, di, and tricarboxylic acids, and/or salts thereof, typically have a molecular weight of less than about 500 g/mol, less than about 400 g/mol, or less than about 300 g/mol.

Non-limiting examples of mono-carboxylic acids, or salts thereof, include formic acid, acetic acid, propionic acid, butyric acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, arachidic acid, lactic acid, a salt thereof, and mixtures of two or more thereof. In some cases, the hair color base or hair color toning compositions include at least lactic acid and/or a salt thereof.

Non-limiting examples of di-carboxylic acids and/or salts thereof include oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, a salt thereof, and mixtures of two or more thereof. In some cases, the hair color base or hair color toning compositions include oxalic acid, malonic acid, malic acid, maleic acid, a salt thereof, or mixtures of two or more thereof.

Non-limiting examples of tricarboxylic acids and salts thereof include citric acid, isocitric acid, aconitric acid, propane-1,2,3-tricarboxylic acid, benzene-1,3,5-tricarboxylic acid, a salt thereof, and mixtures of two or more thereof. In some instances, the hair color base or hair color toning compositions include at least citric acid and/or a salt thereof.

In one or more embodiments, the hair color base or hair color toning composition comprises at least one carboxylic acid chosen from oxalic acid, malonic acid, glutaric acid, succinic acid, adipic acid, glycolic acid, citric acid, tartaric acid, malic acid, sebacic acid, maleic acid, fumaric acid, benzoic acid, citraconic acid, aconitic acid, propane-1,2,3-tricarboxylic acid, trimesic acid, or combinations thereof. In one exemplary embodiment, the compositions include at least citric acid. In a further embodiment, the only carboxylic acid in the composition is citric acid.

The total amount of the at least one carboxylic acid may range from about 0.01% to about 10% by weight, relative to the total weight of the hair color base or hair color toning composition. For example, in some embodiments, the total amount of the at least one carboxylic acid may range from about 0.05% to about 5%, such as about 0.1% to about 5%, about 0.1% to about 4%, about 0.1% to about 3%, about 0.1% to about 2.5%, about 0.1% to about 2.4%, about 0.1% to about 2.3%, about 0.1% to about 2.2%, about 0.1% to about 2.1%, about 0.1% to about 2%, about 0.1% to about 1.9%, about 0.1% to about 1.8%, about 0.1% to about 1.7%, about 0.1% to about 1.6%, about 0.1% to about 1.5%, about 0.1% to about 1.4%, about 0.1% to about 1.3%, about 0.1% to about 1.2%, about 0.1% to about 1.1%, about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, or about 0.1% to about 0.3% by weight, relative to the total weight of the hair color base or hair color toning composition. In other embodiments, the total amount of the at least one carboxylic acid ranges from about 0.5% to about 5%, about 0.5% to about 4%, about 0.5% to about 3%, about 0.5% to about 2.5%, about 0.5% to about 2.4%, about 0.5% to about 2.3%, about 0.5% to about 2.2%, about 0.5% to about 2.1%, about 0.5% to about 2%, about 0.5% to about 1.9%, about 0.5% to about 1.8%, about 0.5% to about 1.7%, about 0.5% to about 1.6%, about 0.5% to about 1.5%, about 0.5% to about 1.4%, about 0.5% to about 1.3%, about 0.5% to about 1.2%, about 0.5% to about 1.1%, about 0.5% to about 1%, about 0.5% to about 0.9%, about 0.5% to about 0.8%, about 0.5% to about 0.7%, or about 0.5% to about 0.6% by weight, relative to the total weight of the hair color base or hair color toning composition.

The total amount of the at least one carboxylic acid may, in certain embodiments, be about 0.05%, about 0.06%, about 0.07%, about 0.08%, about 0.09%, about 0.1%, about 0.11%, about 0.12%, about 0.13%, about 0.14%, about 0.15%, about 0.16%, about 0.17%, about 0.18%, about 0.19%, about 0.2%, about 0.21%, about 0.22%, about 0.23%, about 0.24%, about 0.25%, about 0.26%, about 0.27%, about 0.28%, about 0.29%, about 0.3%, about 0.31%, about 0.32%, about 0.33%, about 0.34%, about 0.35%, about 0.36%, about 0.37%, about 0.38%, about 0.39%, about 0.4%, about 0.41%, about 0.42%, about 0.43%, about 0.44%, about 0.45%, about 0.46%, about 0.47%, about 0.48%, about 0.49%, or about 0.5% by weight, relative to the total weight of the hair color base or hair color toning composition. It is to be understood that any of the above-recited numbers may provide an upper or lower boundary for a range of the total amount of the at least one carboxylic acid.

### Alkalizing Agent(s)

Hair color base and/or hair color toning compositions according to the disclosure may optionally comprise at least one alkalizing agent. The alkalizing agents may be chosen from organic alkalizing agents and/or mineral alkalizing agents. In some embodiments, the hair color base compositions comprise only organic alkalizing agents. In other embodiments, the hair color base compositions comprise only mineral alkalizing agents. In still other embodiments, the hair color base compositions a combination of organic and mineral alkalizing agents.

As non-limiting examples, suitable organic alkalizing agents may be chosen from alkanolamines. In various embodiment, suitable alkanolamines may be chosen from mono-, di- or trialkylamines, comprising one to three identical or different C1-C4 hydroxyalkyl radicals, ethylamines, ethyleneamines, quinoline, aniline and cyclic amines, such as pyrroline, pyrrole, pyrrolidine, imidazole, imidazolidine, imidazolidinine, morpholine, pyridine, piperidine, pyrimidine, piperazine, triazine and derivatives thereof. In some embodiments, alkanolamine alkalizing agents that may be mentioned include but not limited to monoethanolamine (also known as ethanolamine or MEA), diethanolamine, triethanolamine, monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol, 2-amino-2-methyl-1-propanol, and tris(hydroxymethylamino)methane. In some embodiments, the compositions comprise at least one alkalizing agent chosen from alkanolamines, such as monoethanolamine. Optionally, the organic alkalizing agent may comprise, consist essentially of, or consist of alkanolamines, for example monoethanolamine.

As non-limiting examples, suitable mineral alkalizing agents may be chosen from ammonia, ammonium carbonates, sodium carbonates, potassium carbonates, ammonium bicarbonates, sodium bicarbonates, potassium bicarbonates, ammonium hydroxides, sodium hydroxides, potassium hydroxides, salts thereof, hydrates thereof, or mixtures thereof. In some embodiments, the hair color base compositions comprise ammonia and/or ammonium hydroxide. Optionally, the mineral alkalizing agent may consist essentially of or consist of ammonia and/or ammonium hydroxide.

In some embodiments, a hair color base composition disclosed herein comprises at least one organic alkalizing agent and at least one mineral alkalizing agent. In some embodiments, the at least one alkalizing agent comprises, consists essentially of or consists of ammonia or ammonium hydroxide, alkanolamines, salts thereof, hydrates thereof, or combinations thereof. In various embodiments, a hair color base composition disclosed herein comprises both monoethanolamine and ammonium hydroxide.

In some embodiments, alkalizing agents present in a hair color base composition may be only chosen from organic alkalizing agents, and the hair color base composition is free or substantially free of mineral alkalizing agents, such as ammonia and/or ammonium hydroxide. In this case, the hair color base composition may comprise less than about 0.5%, less than about 0.4%, less than about 0.3%, less than about 0.2%, less than about 0.1%, or less than about 0.05% of mineral alkalizing agents. In certain embodiments, the hair color base compositions disclosed herein comprise one or more organic alkalizing agents and are free or substantially free of ammonia and/or ammonium-based compounds.

In various embodiments, the amount of organic or mineral alkalizing agents in the hair color base or hair color toning compositions may independently range from about 0.1% to about 10%, such as from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 0.5% to about 1%, from about 0.75% to about 10%, from about 0.75% to about 9%, from about 0.75% to about 8%, from about 0.75% to about 7%, from about 0.75% to about 6%, from about 0.75% to about 5%, from about 0.75% to about 4%, from about 0.75% to about 3%, from about 0.75% to about 2%, from about 0.75% to about 1%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 2% to about 10%, from about 2% to about 9%, from about 2% to about 8%, from about 2% to about 7%, from about 2% to about 6%, from about 2% to about 5%, from about 2% to about 4%, from about 2% to about 3%, from about 3% to about 10%, from about 3% to about 9%, from about 3% to about 8%, from about 3% to about 7%, from about 3% to about 6%, from about 3% to about 5%, from about 3% to about 4%, from about 4% to about 10%, from about 4% to about 9%, from about 4% to about 8%, from about 4% to about 7%, from about 4% to about 6%, from about 4% to about 5%, from about 5% to about 10%, from about 5% to about 9%, from about 5% to about 8%, from about 5% to about 7%, or from about 5% to about 6% by weight, based on the total weight of the hair color base or hair color toning composition.

For example, in some embodiments, the alkalizing agents may comprise at least one organic alkalizing agent and at least one mineral alkalizing agent, wherein the at least one organic alkalizing agent is present in an amount ranging from about 0.1% to about 5%, such as from about 0.2% to about 4%, from about 0.3% to about 3%, from about 0.4% to about 2%, or from about 0.5% to about 1.5% by weight, relative to the total weight of the hair color base composition; and the at least one mineral alkalizing agent is present in an amount ranging from about 0.5% to about 10%, such as from about 1% to about 8%, from about 2% to about 6%, or from about 3% to about 5% by weight, relative to the total weight of the hair color base or hair color toning composition. For instance, the composition may comprise at least one alkanolamine, e.g. monoethanolamine, in combination with ammonia and/or ammonium hydroxide, in the above-mentioned ranges.

In some embodiments, the hair color base or hair color toning compositions according to the disclosure comprise monoethanolamine.

In various embodiments, hair color base or hair color toning compositions according to the disclosure comprise monoethanolamine in an amount up to about 10%, such from about 0.001% up to about 10%, or from about 0.01% up to about 5%, up to about 4%, up to about 3.9%, up to about 3.8%, up to about 3.7%, up to about 3.6%, up to about 3.5%, up to about 3.4%, up to about 3.3%, up to about 3.2%, up to about 3.1%, up to about 3%, up to about 2.9%, up to about 2.8%, up to about 2.7%, up to about 2.6%, up to about 2.5%, up to about 2.4%, up to about 2.3%, up to about 2.2%, up to about 2.1%, up to about 2.0%, up to about 1.9%, up to about 1.8%, up to about 1.7%, up to about 1.6%, up to about 1.5%, up to about 1.4%, up to about 1.3%, up to about 1.2%, up to about 1.1%, up to about 1%, up to about 0.9%, up to about 0.8%, up to about 0.7%, up to about 0.6%, up to about 0.5%, up to about 0.4%, up to about 0.3%, up to about 0.2%, up to about 0.1%, or up to about 0.05% by weight, based on the weight of the composition. By way of example, the monoethanolamine may be present in an amount of about 0.1%, about 0.11%, about 0.12%, about 0.13%, about 0.14%, about 0.15%, about 0.16%, about 0.17%, about 0.18%, about 0.19%, about 0.2%, about 0.21%, about 0.22%, about 0.23%, about 0.24%, about 0.25%, about 0.26%, about 0.27%, about 0.28%, about 0.29%, about 0.3%, about 0.31%, about 0.32%, about 0.33%, about 0.34%, about 0.35%, about 0.36%, about 0.37%, about 0.38%, about 0.39%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, or about 3.5% by weight, based on the weight of the composition, including all ranges and subranges using any of the foregoing as upper and lower limits.

In various embodiments, hair color base or hair color toning compositions according to the disclosure comprise ammonia in an amount up to about 2% to about 20% by weight, such as from about 2% to about 11% by weight, and further such as from about 4.5% to about 10% by weight, based on the total weight of the hair color composition, including increments and ranges therein there between.

The total amount of ammonia may be employed in an amount of from about 2%, 3%, 4%, 5%, 6%, 7%, 8%, 9%, 10%, 11%, 12%, 13%, 14%, 15%, 16%, 17%, 18%, 19%, to about 20% by weight, including increments and ranges therein there between, based on the weight of the composition including all ranges and subranges using any of the foregoing as upper and lower limits.

In various embodiments, hair color base or hair color toning compositions according to the disclosure contain a small amount (i.e., greater than 0 but less than 2, 1, or 0.5% by weight) of ammonia, or is substantially free of ammonia.

### Fatty Compound(s)

Fatty compounds (also referred to interchangeably as “fatty substances”) may optionally be included in hair color base and/or hair color toning compositions of the disclosure. In some embodiments, two or more fatty compounds may be included. In further embodiments, such fatty compounds may be chosen from fatty compounds other than a fatty acid. As used herein, “fatty compound” means an organic compound insoluble in water at normal temperature (25° C.) and at atmospheric pressure (760 mmHg) (solubility below 5% and such as below 1% and further such as below 0.1%). Fatty compounds have in their structure a chain of at least two siloxane groups or at least one hydrocarbon chain having at least 6 carbon atoms. Moreover, fatty compounds are generally soluble in organic solvents in the same conditions of temperature and pressure, for example in chloroform, ethanol, benzene or decamethylcyclopentasiloxane.

If present, the total amount fatty compounds is generally less than about 35%, for example may range from about 0.1% to about 30%, such as from about 0.5% to about 30%, from about 1% to about 30%, from about 5% to about 30%, from about 10% to about 30%, from about 15% to about 30%, from about 20% to about 30%, from about 0.1% to about 25%, from about 0.5% to about 25%, from about 1% to about 25%, from about 5% to about 25%, from about 10% to about 25%, from about 15% to about 25%, or from about 20% to about 25% by weight, relative to the total weight of the hair color base or hair color toning composition.

Non-limiting examples of useful fatty compounds include lower alkanes, fatty alcohols, esters of fatty acid, esters of fatty alcohol, oils such as mineral, vegetable, animal and synthetic non-silicone oils, non-silicone waxes, and silicones.

In some embodiments, the alcohols and esters have at least one linear or branched, saturated or unsaturated hydrocarbon group, comprising 6 to 30 carbon atoms, optionally substituted, for example, with at least one hydroxyl group (for example 1 to 4). If they are unsaturated, these compounds can have one to three, conjugated or unconjugated, carbon-carbon double bonds.

With regard to the lower alkanes, in some embodiments, these have from 6 to 16 carbon atoms and are linear or branched, optionally cyclic. As examples, alkanes can be chosen from hexane and dodecane, isoparaffins such as isohexadecane and isodecane.

Non-limiting examples of non-silicone oils usable in the composition of the disclosure, include: esters of a glycerol oligomer, in particular diglycerol esters, especially condensates of adipic acid and of glycerol, for which a portion of the hydroxyl groups of the glycerols has reacted with a mixture of fatty acids, such as stearic acid, capric acid, isostearic acid and 12-hydroxystearic acid, such as in particular those sold under the brand name Softisan 649 by Sasol; arachidyl propionate, sold under the brand name Waxenol 801 by Alzol; fatty acid triglycerides and their derivatives; pentaerythritol esters; esters of dimer diol and dimer diacid, if appropriate esterified on their free alcohol or acid functional group(s) by acid or alcohol radicals, in particular dimer dilinoleate esters; such esters can be chosen in particular from esters with the following INCI nomenclature: bis-behenyl/isostearyl/phytosteryl dimer dilinoleyl dimer dilinoleate (Plandool G), phytosteryl isostearyl dimer dilinoleate (Lusplan PI-DA or Lusplan PHY/IS-DA), phytosteryl/isostearyl/cetyl/stearyl/behenyl dimer dilinoleate (Plandool H or Plandool S), and their mixtures; mango butter, such as that sold under the reference Lipex 203 by AarhusKarlshamn; hydrogenated soybean oil, hydrogenated coconut oil, hydrogenated rapeseed oil or mixtures of hydrogenated vegetable oils, such as the soybean, coconut, palm and rapeseed hydrogenated vegetable oil mixture, for example the mixture sold under the reference Akogel® by AarhusKarlshamn (INCI name: Hydrogenated Vegetable Oil); shea butter, in particular that having the INCI name Butyrospermum Parkii Butter, such as that sold under the reference Sheasoft® by AarhusKarlshamn; cocoa butter, in particular that which is sold under the name CT Cocoa Butter Deodorized by Dutch Cocoa BV or that which is sold under the name Beurre De Cacao NCB HD703 758 by Barry Callebaut; shorea butter, in particular that which is sold under the name Dub Shorea T by Stearinerie Dubois; and their mixtures.

In some embodiments, the fatty compound is chosen from hydrogenated vegetable oil, shea butter, cocoa butter, shorea butter, a soybean, coconut, palm and rapeseed hydrogenated vegetable oil mixture, and their mixtures.

Non-limiting examples of non-silicone oils usable in the composition of the disclosure, include: hydrocarbon oils of animal origin, such as perhydrosqualene; hydrocarbon oils of vegetable origin, such as liquid triglycerides of fatty acids having from 6 to 30 carbon atoms such as triglycerides of heptanoic or octanoic acids, or for example sunflower oil, maize oil, soya oil, cucurbit oil, grapeseed oil, sesame oil, hazelnut oil, apricot oil, macadamia oil, arara oil, sunflower oil, castor oil, avocado oil, triglycerides of caprylic/capric acids such as those sold by the company Stearineries Dubois or those sold under the names MIGLYOL® 810, 812 and 818 by the company Dynamit Nobel, jojoba oil, shea butter oil; hydrocarbons with more than 16 carbon atoms, linear or branched, of mineral or synthetic origin, such as paraffin oils, petroleum jelly, liquid paraffin, polydecenes, hydrogenated polyisobutene such as Parleam®. fluorinated, partially hydrocarbon oils; as fluorinated oils, non-limiting examples include perfluoromethylcyclopentane and perfluoro-1,3-dimethylcyclohexane, sold under the names “FLUTEC® PC1” and “FLUTEC® PC3” by the company BNFL Fluorochemicals; perfluoro-1,2-dimethylcyclobutane; perfluoroalkanes such as dodecafluoropentane and tetradecafluorohexane, sold under the names “PF 5050®” and “PF 5060®” by the 3M Company, or bromoperfluorooctyl sold under the name “FORALKYL®” by the company Atochem; nonafluoro-methoxybutane and nonafluoroethoxyisobutane; derivatives of perfluoromorpholine, such as 4-trifluoromethyl perfluoromorpholine sold under the name “PF 5052®” by the 3M Company. The non-silicone oils may be employed in an amount of from about 0.5% to about 5% by weight, such as from about 1% to about 5.5% by weight, and further such as from about 1.5% to about 4% by weight, based on the total weight of the hair color base or hair color toning composition, including increments and ranges therein there between. For example, the total amount of non-silicone oils may range from about 0.5%, 0.6%, 0.7%, 0.8%, 0.9%, 1%, 2%, 3%, or 4% to about 5% by weight of the composition, including increments and ranges therein there between.

As used herein, “fatty alcohol” refers to any alcohol with a carbon chain of C5 or greater, such as, for example, C8 or greater, C10 or greater, and C12 or greater. The at least one fatty alcohol may be chosen from, for example, C9-C11 alcohols, C12-C13 alcohols, C12-C15 alcohols, C12-C16 alcohols, C14-C15 alcohols, C12-C22 alcohols, arachidyl alcohol, behenyl alcohol, caprylic alcohol, cetearyl alcohol, cetyl alcohol, coconut alcohol, decyl alcohol, hydrogenated tallow alcohol, jojoba alcohol, lauryl alcohol, myristyl alcohol, oleyl alcohol, palm alcohol, palm kernel alcohol, stearyl alcohol, tallow alcohol, and tridecyl alcohol.

As used herein, “alkoxylated fatty alcohol” refers to any fatty alcohol with a carbon chain of C5 or greater, as defined above, further comprising at least one alkoxy group. For example, the at least one alkoxylated fatty alcohol may have a carbon chain of C8 or greater, C10 or greater, and C12 or greater. Further, for example, the at least one alkoxylated fatty alcohol may be chosen from alkoxylated polymers (including co-, ter- and homo-polymers) derived from alcohols such as glycerol (e.g. polyglyceryl derived from four glycerol molecules). The at least one alkoxy group of the at least one alkoxylated fatty alcohol may, for example, be derived from an alkoxylation reaction carried out with alkylene oxide. Non-limiting examples of at least one alkoxylated fatty alcohol include any fatty alcohol comprising at least one polyethylene glycol ether and any fatty alcohol comprising at least one polypropylene glycol ether.

Non-limiting examples of the at least one alkoxylated fatty alcohol include ceteareth-2, ceteareth-3, ceteareth-4, ceteareth-5, ceteareth-6, ceteareth-7, ceteareth-8, ceteareth-9, ceteareth-10, ceteareth-11, ceteareth-12, ceteareth-13, ceteareth-14, ceteareth-15, ceteareth-16, ceteareth-17, ceteareth-18, ceteareth-20, ceteareth-22, ceteareth-23, ceteareth-24, ceteareth-25, ceteareth-27, ceteareth-28, ceteareth-29, ceteareth-30, cetearel:h-33, ceteareth-34, ceteareth-40, ceteareth-50, ceteareth-55, ceteareth-60, ceteareth-80, ceteareth-100, laureth-1, laureth-2, laureth-3, laureth-4, laureth-5, laureth-6, laureth-7, laureth-8, laureth-9, laureth-10, laureth-11, laureth-12, laureth-13, laureth-14, laureth-15, lauretih-16, laureth-20, laureth-23, laureth-25, laureth-30, laureth-40, deceth-3, deceth-5, oleth-5, oleth-30, steareth-2, steareth-10, steareth-20, steareth-100, cetylsteareth-12, ceteareth-5, ceteareth-5, polyglyceryl 4-lauryl ether, polyglyceryl 4-oleyl ether, polyglyceryl 2-oleyl ether, polyglyceryl 2-cetyl ether, polyglyceryl 6-cetyl ether, polyglyceryl 6-oleylcetyl ether, polyglyceryl 6-octadecyl ether, C9-C11 pareth-3, C9-C11 pareth-6, C11-C15 pareth-3, C11-C15 pareth-5, C11-C15 pareth-12, C 11-C15 pareth-20, C12-C15 pareth-9, C12-C15 pareth-12, and C22-C24 pareth-33.

The fatty alcohols useful according to the disclosure include, but are not limited to, non-alkoxylated, saturated or unsaturated, linear or branched, and have from 6 to 30 carbon atoms and more particularly from 8 to 30 carbon atoms; For example, cetyl alcohol, stearyl alcohol and their mixture (cetylstearyl alcohol), octyldodecanol, 2-butyloctanol, 2-hexyldecanol, 2-undecylpentadecanol, oleic alcohol or linoleic alcohol.

If present, the total amount of fatty alcohols may range from about 0.1% to about 15%, such as from about 0.5% to about 15%, from about 1% to about 15%, from about 2% to about 15%, from about 3% to about 15%, from about 4% to about 15%, from about 5% to about 15%, from about 6% to about 15%, from about 7% to about 15%, from about 8% to about 15%, from about 9% to about 15%, from about 10% to about 15%, from about 0.1% to about 10%, from about 0.5% to about 10%, from about 1% to about 10%, from about 2% to about 10%, from about 3% to about 10%, from about 4% to about 10%, from about 5% to about 10%, from about 6% to about 10%, from about 7% to about 10%, or from about 8% to about 10% by weight, relative to the total weight of the composition.

The exemplary non-silicone wax or waxes that can be used in the composition of the disclosure are chosen from carnauba wax, candelilla wax, and Alfa wax, paraffin wax, ozokerite, vegetable waxes such as olive wax, rice wax, hydrogenated jojoba wax or absolute waxes of flowers such as the essential wax of blackcurrant flower sold by the company BERTIN (France), animal waxes such as beeswaxes, or modified beeswaxes (cerabellina); other waxes or waxy raw materials usable according to the disclosure are, for example, marine waxes such as that sold by the company SOPHIM under reference M82, waxes of polyethylene or of polyolefins in general.

The exemplary fatty acid esters are the esters of saturated or unsaturated, linear or branched C1-C26 aliphatic mono- or polyacids and of saturated or unsaturated, linear or branched C1-C26 aliphatic mono- or polyalcohols, the total number of carbons of the esters being, for example, greater than or equal to 10.

Among the monoesters, non-limiting mentions can be made of dihydroabietyl behenate; octyldodecyl behenate; isocetyl behenate; cetyl lactate; C12-C15 alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, ethyl-2-hexyl palmitate, 2-octyldecyl palmitate, alkyl myristates such as isopropyl, butyl, cetyl, 2-octyldodecyl, mirystyl, stearyl myristate, hexyl stearate, butyl stearate, isobutyl stearate; dioctyl malate, hexyl laurate, and 2-hexyldecyl laurate.

Further non-limiting mentions of esters can be made of the esters of C4-C22 di- or tricarboxylic acids and of C1-C22 alcohols and the esters of mono-, di- or tricarboxylic acids and of C2-C26 di-, tri-, tetra- or pentahydroxy alcohols.

Even further non-limiting examples of esters include: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate, tridecyl erucate; triisopropyl citrate; triisotearyl citrate; glyceryl trilactate; glyceryl trioctanoate; trioctyldodecyl citrate; trioleyl citrate, propylene glycol dioctanoate; neopentyl glycol diheptanoate; diethylene glycol diisanonate; glycol distearates; and polyethylene glycol distearates.

Among the esters mentioned above, exemplary esters include ethyl, isopropyl, myristyl, cetyl, stearyl palmitates, ethyl-2-hexyl palmitate, 2-octyldecyl palmitate, alkyl myristates such as isopropyl, butyl, cetyl, 2-octyldodecyl myristate, hexyl stearate, butyl stearate, isobutyl stearate; dioctyl malate, hexyl laurate, 2-hexyldecyl laurate and isononyl isononanate, cetyl octanoate.

The composition can also comprise, as fatty ester, esters and di-esters of sugars of C6-C30, such as C12-C22 fatty acids. “Sugar” as used in the disclosure means oxygen-containing hydrocarbon compounds that possess several alcohol functions, with or without aldehyde or ketone functions, and having at least 4 carbon atoms. These sugars can be monosaccharides, oligosaccharides or polysaccharides.

As suitable sugars, non-limiting examples include sucrose, glucose, galactose, ribose, fucose, maltose, fructose, mannose, arabinose, xylose, lactose, and their derivatives, for example alkylated, such as methylated derivatives such as methylglucose.

The esters of sugars and of fatty acids can, for example, be chosen from the esters or mixtures of esters of sugars described previously and of linear or branched, saturated or unsaturated C6-C30, such as C12-C22 fatty acids. If they are unsaturated, these compounds can have one to three, conjugated or unconjugated, carbon-carbon double bonds.

The esters according to some embodiments can be chosen from mono-, di-, tri- and tetra-esters, polyesters and mixtures thereof.

These esters can be for example oleate, laurate, palmitate, myristate, behenate, cocoate, stearate, linoleate, linolenate, caprate, arachidonates, or mixtures thereof such as the oleo-palmitate, oleo-stearate, palmito-stearate mixed esters.

For example, the mono- and di-esters can be used, and such as the mono- or di-oleate, stearate, behenate, oleopalmitate, linoleate, linolenate, oleostearate, of sucrose, of glucose or of methylglucose.

Non-limiting mention can be made of the product sold under the name GLUCATE® DO by the company Amerchol, which is a dioleate of methylglucose.

Exemplary esters or of mixtures of esters of sugar of fatty acid include: the products sold under the names F160, F140, F110, F90, F70, SL40 by the company Crodesta, denoting respectively the palmitostearates of sucrose formed from 73% of monoester and 27% of di- and tri-ester, from 61% of monoester and 39% of di-, tri-, and tetra-ester, from 52% of monoester and 48% of di-, tri-, and tetra-ester, from 45% of monoester and 55% of di-, tri-, and tetra-ester, from 39% of monoester and 61% of di-, tri-, and tetra-ester, and the mono-laurate of sucrose; the products sold under the name Ryoto Sugar Esters for example with the reference B370 and corresponding to the behenate of sucrose formed from 20% of monoester and 80% of di-triester-polyester; sucrose mono-di-palmito-stearate marketed by the company Goldschmidt under the name TEGOSOFT® PSE.

### Surfactants

The hair color base and/or hair color toning compositions optionally comprise one or more surfactants chosen from anionic surfactants, nonionic surfactants, amphoteric surfactants, and mixtures thereof. In at least one exemplary embodiment, the surfactant is a surfactant mixture comprising at least one amphoteric surfactant and at least one anionic surfactant. In a further embodiment, the surfactant is a surfactant mixture comprising at least one amphoteric surfactant and at least one non-ionic surfactant. In yet a further embodiment, the surfactant is a surfactant mixture comprising at least one non-ionic surfactant and at least one anionic surfactant.

The total amount of the one or more surfactants included in the hair color base or hair color toning compositions can vary, especially depending on the type of hair color base or hair color toning composition in with they are contained. The total amount of the one or more surfactants typically ranges from about 0.1% to about 60% by weight, relative to the total weight of the hair color base or hair color toning composition, including all ranges and subranges therebetween. In some cases, the total amount of the one or more surfactants ranges from about 0.1% to about 50%, about 0.1% to about 45%, about 0.1% to about 40%, about 0.1% to about 35%, about 0.1% to about 30%, about 0.1 to about 25%, about 0.1 to about 20%, about 0.1 to about 15%, about 0.1 to about 10%, about 0.1 to about 5%, about 0.5 to about 40%, about 0.5 to about 35%, about 0.5 to about 30%, about 0.5 to about 25%, about 0.5 to about 20%, about 0.5 to about 15%, about 0.5 to about 10%, about 0.5 to about 5%, about 1% to about 40%, about 1% to about 30%, about 1% to about 20%, about 1% to about 15%, about 1% to about 10%, or about 1% to about 5% by weight, relative to the total weight of the hair treatment composition. In further embodiments, the total amount of the one or more surfactants ranges from about trimester 15% to about 55%, about 15% to about 50%, about 15% to about 45%, about 15% to about 40%, about 15% to about 35%, about 15% to about 30%, about 15% to about 25%, about 20% to about 55%, about 20% to about 50%, about 20% to about 45%, about 20% to about 40%, about 20% to about 35%, about 20% to about 30%, about 20% to about 25%, about 25% to about 55%, about 25% to about 50%, about 25% to about 45%, about 25% to about 40%, about 25% to about 35%, about 25% to about 30%, 30% to about 55%, about 30% to about 50%, about 30% to about 45%, about 30% to about 40%, about 30% to about 35%, about 35% to about 55%, about 35% to about 50%, about 35% to about 45%, about 35% to about 40%, about 40% to about 55%, about 40% to about 50%, or about 40% to about 45% by weight, relative to the total weight of the hair color base or hair color toning composition.

### Anionic Surfactants

The term “anionic surfactant” means a surfactant comprising, as ionic or ionizable groups, only anionic groups. These anionic groups may optionally be chosen from the groups CO₂H, CO₂⁻, SO₃H, SO₃⁻, OSO₃H, OSO₃⁻ O₂PO₂H, O₂PO₂H and O₂PO₂²⁻.

The hair color base or hair color toning compositions may include one or more anionic surfactants. Non-limiting examples of anionic surfactants include alkyl sulfates, alkyl ether sulfates, acyl isethionates, acyl glycinates, acyl taurates, acyl amino acids, acyl sarcosinates, sulfosuccinates, sulfonates, and mixtures of two or more thereof, wherein the alkyl and acyl groups of all these compounds comprise from 6 to 24 carbon atoms. In some cases, anionic sulfate surfactants may be excluded from the one or more anionic surfactants. In such cases, the one or more anionic surfactants may be chosen from acyl isethionates, acyl glycinates, acyl taurates, acyl amino acids, acyl sarcosinates, sulfosuccinates, sulfonates, and mixtures of two or more thereof, wherein the alkyl and acyl groups of all these compounds comprise from 6 to 24 carbon atoms.

The total amount of the one or more anionic surfactants may be about 1 to about 40% by weight, relative to the total weight of the hair color base or hair color toning composition, including all ranges and subranges therebetween. Furthermore, the total amount of the one or more anionic surfactants may be about 1 to about 35%, about 1 to about 30%, about 5% to about 40%, about 5% to about 25%, about 5% to about 30%, about 10% to about 40%, about 10% to about 35%, or about 15% to about 40%.

The anionic surfactant(s) that may be used may be alkyl sulfates, alkyl ether sulfates, alkylamido ether sulfates, alkylaryl polyether sulfates, monoglyceride sulfates, alkylsulfonates, alkylamide sulfonates, alkylarylsulfonates, alpha-olefin sulfonates, paraffin sulfonates, alkylsulfosuccinates, alkyl ether sulfosuccinates, alkylamide sulfosuccinates, alkyl sulfoacetates, acylsarcosinates, acylglutamates, alkylsulfosuccinamates, acylisethionates and N-acyltaurates, salts of alkyl monoesters and polyglycoside-polycarboxylic acids, acyllactylates, salts of D-galactoside uronic acids, salts of alkyl ether carboxylic acids, salts of alkyl aryl ether carboxylic acids, and salts of alkylamido ether carboxylic acids; or the non-salified forms of all of these compounds, the alkyl and acyl groups of all of these compounds containing from 6 to 24 carbon atoms and the aryl group denoting a phenyl group. Some of these compounds may be oxyethylenated and then preferably comprise from 1 to 50 ethylene oxide units. For example, the anionic surfactant may be chosen from sodium olefin sulfonates, e.g. sodium C14-C16 olefin sulfonate.

The salts of C₆-C₂₄ alkyl monoesters of polyglycoside-polycarboxylic acids may be chosen from C₆-C₂₄ alkyl polyglycoside-citrates, C₆-C₂₄ alkyl polyglycoside-tartrates and C₆-C₂₄ alkyl polyglycoside-sulfo succinates.

When the anionic surfactant(s) are in salt form, they may be chosen especially from alkali metal salts such as the sodium or potassium salt and preferably the sodium salt, ammonium salts, amine salts and in particular amino alcohol salts, or alkaline-earth metal salts such as the magnesium salt.

Examples of amino alcohol salts that may especially be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts. Alkali metal or alkaline-earth metal salts and in particular the sodium or magnesium salts may be used.

Use is also made of (C₆-C₂₄)alkyl sulfates, (C₆-C₂₄)alkyl ether sulfates, which are optionally ethoxylated, comprising from 2 to 50 ethylene oxide units, and mixtures of two or more thereof, in particular in the form of alkali metal salts or alkaline-earth metal salts, ammonium salts or amino alcohol salts. More preferentially, the anionic surfactant(s) are chosen from (C₁₀-C₂₀)alkyl ether sulfates, and in particular sodium lauryl ether sulfate.

### Non-Ionic Surfactants

Non-ionic surfactants are compounds well known in themselves (see, e.g., in this regard, “Handbook of Surfactants” by M. R. Porter, Blackie & Son publishers (Glasgow and London), 1991, pp. 116-178), which is incorporated herein by reference in its entirety.

The total amount of the one or more non-ionic surfactants may be about 1 to about 40% by weight, relative to the total weight of the hair color base or hair color toning composition, including all ranges and subranges therebetween. Furthermore, the total amount of the one or more non-ionic surfactants may be about 1 to about 35%, about 1 to about 30%, about 5% to about 40%, about 5% to about 25%, about 5% to about 30%, about 10% to about 40%, about 10% to about 35%, or about 15% to about 40%.

The non-ionic surfactant can be, for example, chosen from alcohols, alpha-diols, alkylphenols and esters of fatty acids, these compounds being ethoxylated, propoxylated or glycerolated and having at least one fatty chain comprising, for example, from 8 to 18 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives, amine oxides such as (C₁₀-C₁₄)alkylamine oxides or N—(C₁₀-C₁₄)acylaminopropylmorpholine oxides; and mixtures of two or more thereof.

The nonionic surfactants may preferably be chosen from polyoxyalkylenated or polyglycerolated nonionic surfactants. The oxyalkylene units are more particularly oxyethylene or oxypropylene units, or a mixture thereof, and are preferably oxyethylene units.

Examples of oxyalkylenated nonionic surfactants that may be mentioned include: oxyalkylenated (C₈-C₂₄)alkylphenols, saturated or unsaturated, linear or branched, oxyalkylenated C₈-C₃₀ alcohols, saturated or unsaturated, linear or branched, oxyalkylenated C₈-C₃₀ amides, esters of saturated or unsaturated, linear or branched, C₈-C₃₀ acids and of polyethylene glycols, polyoxyalkylenated esters of saturated or unsaturated, linear or branched, C₈-C₃₀ acids and of sorbitol, saturated or unsaturated, oxyalkylenated plant oils, condensates of ethylene oxide and/or of propylene oxide, inter alia, alone or as mixtures.

As examples of polyglycerolated nonionic surfactants, polyglycerolated C₈-C₄₀ alcohols are preferably used. In particular, the polyglycerolated C₈-C₄₀ alcohols correspond to the following formula:

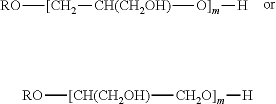

in which R represents a linear or branched C₈-C₄₀ and preferably C₈-C₃₀ alkyl or alkenyl radical, and m represents a number ranging from 1 to 30 and preferably from 1.5 to 10.

As non-limiting examples, mention may be made of lauryl alcohol containing 4 mol of glycerol (INCI name: Polyglyceryl-4 Lauryl Ether), lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

According to some embodiments, the nonionic surfactant may be chosen from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and mixtures of two or more thereof.

Preferably, the nonionic surfactant may be a nonionic surfactant with an HLB of 18.0 or less, such as from 4.0 to 18.0, more preferably from 6.0 to 15.0 and furthermore preferably from 9.0 to 13.0. The HLB is the ratio between the hydrophilic part and the lipophilic part in the molecule. This term HLB is well known to those skilled in the art and is described in “The HLB system. A time-saving guide to emulsifier selection” (published by ICI Americas Inc., 1984).

In some case, the nonionic surfactant is a fatty alkanolamide. Non-limiting examples of fatty alkanolamides that may be used include cocamide MEA, cocamide DEA, soyamide DEA, lauramide DEA, oleamide MIPA, stearamide MEA, myristamide DEA, stearamide DEA, oleylamide DEA, tallowamide DEA lauramide MIPA, tallowamide MEA, isostearamide DEA, isostearamide MEA, and mixtures of two or more thereof.

### Amphoteric Surfactants

Compositions according to the disclosure may comprise at least one amphoteric surfactant. Non-limiting examples of amphoteric surfactants useful in the compositions include, for example, optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

Mention may be made in particular of (C8-C20)alkylbetaines, sulfobetaines, (C8-C20)alkylsulfobetaines, (C8-C20)alkylamido(C1-C6)alkylbetaines, such as cocamidopropylbetaine, and (C8-C20)alkylamido(C1-C6)alkylsulfobetaines, and mixtures thereof. For example, mention may be made of compounds classified under the INCI names sodium cocoamphoacetate, sodium lauroamphoacetate, sodium caproamphoacetate and sodium capryloamphoacetate.

Other compounds that may be chosen include disodium cocoamphodiacetate, disodium lauroamphodiacetate, disodium caproamphodiacetate, disodium capryloamphodiacetate, disodium cocoamphodipropionate, disodium lauroamphodipropionate, disodium caproamphodipropionate, disodium capryloamphodipropionate, lauroamphodipropionic acid and cocoamphodipropionic acid.

Examples that may be mentioned include the cocoamphodiacetate sold by the company Rhodia under the trade name Miranol®. C2M Concentrate, the sodium cocoamphoacetate sold under the trade name Miranol Ultra C 32 and the product sold by the company Chimex under the trade name CHIMEXANE HA. In certain exemplary embodiments, the amphoteric surfactants are chosen from (C8-C20)alkylbetaines such as the one known under the INCI names coco-betaine, (C8-C20)alkylamido(C1-C6)alkylbetaines such as the one known under the INCI name cocamidopropylbetaine, and mixtures thereof. In various embodiments, the amphoteric surfactant is coco-betaine.

The compositions may comprise amphoteric surfactant(s) in an amount ranging from about 0.1% to about 10%, such as from about 0.5% to about 8%, from about 1% to about 5%, or from about 1% to about 3% by weight, relative to the total weight of the composition.

### Solvent

The hair color base or hair color toning compositions according to the disclosure comprise a solvent. The solvent may be chosen from water, non-aqueous solvents, or combinations thereof. The solvent may be present in the hair color base or hair color toning composition in an amount ranging from about 10% to about 95% by weight, relative to the total weight of the hair color base or hair color toning composition. For example, the total amount of solvent may range from about 20% to about 90%, about 20% to about 85%, about 20% to 75%, about 20% to 70%, about 20% to about 65%, about 20% to about 60%, about 20% to about 55%, about 30% to about 90%, about 30% to about 85%, about 30% to 75%, about 30% to 70%, about 30% to about 65%, about 30% to about 60%, about 30% to about 55%, about 40% to about 90%, about 40% to about 85%, about 40% to 75%, about 40% to 70%, about 40% to about 65%, about 40% to about 60%, about 40% to about 55%, about 50% to about 90%, about 50% to about 85%, about 50% to 75%, about 50% to 70%, about 50% to about 65%, or about 50% to about 60% by weight, relative to the total weight of the hair color base or hair color toning composition.

In some embodiments, the solvent comprises, consists essentially of, or consists of water. The total amount of water in the hair color base or hair color toning compositions may vary depending on the type of composition and the desired consistency, viscosity, etc. In some embodiments, the total amount of water is about 10% to about 95% by weight, relative to the total weight of the hair color base or hair color toning composition, including all ranges and subranges therebetween. For example, the total amount of water may be about 10% to about 90%, about 10% to about 85%, about 10% to 75%, about 10% to 70%, about 10% to about 65%, about 10% to about 60%, about 10% to about 55%, about 10% to about 50%, about 10% to about 45%, about 10% to about 40%, about 10% to about 35%, about 10% to about 30%, or about 15% to about 30% by weight, relative to the total weight of the hair color base or hair color toning composition. It may, in at least certain embodiments, be desirable to include water in an amount less than about 60%, less than about 55%, less than about 50%, less than about 45%, less than about 40%, less than about 35%, less than about 30%, less than about 25%, or less than about 20% by weight, relative to the total weight of the hair color base or hair color toning composition. For example, the water may be present in an amount ranging from about 1% to about 55%, about 1% to about 50%, about 1% to about 45%, about 1% to about 40%, about 1% to about 35%, about 1% to about 30%, about 1% to about 25%, about 1% to about 20%, 5% to about 55%, about 5% to about 50%, about 5% to about 45%, about 5% to about 40%, about 5% to about 35%, about 5% to about 30%, about 5% to about 25%, about 5% to about 20%, 10% to about 55%, about 10% to about 50%, about 10% to about 45%, about 10% to about 40%, about 10% to about 35%, about 10% to about 30%, about 10% to about 25%, about 10% to about 20%, 15% to about 55%, about 15% to about 50%, about 15% to about 45%, about 15% to about 40%, about 15% to about 35%, about 15% to about 30%, about 15% to about 25%, or about 15% to about 20% by weight, relative to the total weight of the hair color base or hair color toning composition.

In certain embodiments, the composition comprises, consists essentially of, or consists of non-aqueous solvents, for example, glycerin, C₁-4 alcohols, organic solvents, fatty alcohols, fatty ethers, fatty esters, polyols, glycols, vegetable oils, mineral oils, liposomes, laminar lipid materials, or any mixtures of two or more thereof. As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of solvents which may be used include alkanediols (polyhydric alcohols) such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and mixtures of two or more thereof.

In some cases, the water-soluble solvent may be chosen from glycols, C₁-4 alcohols, glycerin, and mixtures thereof. In some cases, the water-soluble solvent is chosen from hexylene glycol, proplene glycol, caprylyl glycol, glycerin, isopropyl alcohol, and mixtures of two or more thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and mixtures of two or more thereof.

Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and mixtures of two or more thereof.

The total amount of the non-aqueous solvents may vary, but in some cases ranges from about 0.01% to about 50% by weight, relative to the total weight of the hair color base or hair color toning composition. For example, the total amount of non-aqueous solvents may range from about 1% to about 50%, about 2% to about 50%, about 3% to 50%, about 4% to about 50%, about 5% to about 50%, 1% to about 40%, about 2% to about 40%, about 3% to 40%, about 4% to about 40%, about 5% to about 40%, about 1% to about 35%, about 2% to about 35%, about 3% to 35%, about 4% to about 35%, or about 5% to about 35% by weight, relative to the total weight of the composition. In certain embodiments, the total amount of non-aqueous solvents may range from about 1% to about 10%, about 2% to about 8%, about 3% to about 7%, about 25% to about 45%, about 25% to about 40%, about 25% to about 35%, or about 30% to about 35% by weight, relative to the total weight of the composition.

### Thickening Agent(s)

In some embodiments, the hair color base and/or hair color toning compositions optionally further comprise a thickening agent. In other embodiments, the hair color base and/or hair color toning compositions do not comprise a thickening agent.

Non-limiting examples of thickening agents include, but are not limited to, viscous liquids, such as polyethylene glycol, semisynthetic polymers, such as semisynthetic cellulose derivatives, synthetic polymers, such as carbomers, poloxamers, and acrylates/beheneth-25 methacrylate copolymer, acrylates copolymer, polyethyleneimines (e.g., PEI-10), naturally occurring polymers, such as acacia, tragacanth, alginates (e.g., sodium alginate), carrageenan, vegetable gums, such as xanthan gum, petroleum jelly, waxes, particulate associate colloids, such as bentonite, colloidal silicon dioxide, and microcrystalline cellulose, surfactants, such as PPG-2 hydroxyethyl coco/isostearamide, emulsifiers, such as disteareth-75 IPDI, and salts, such as sodium chloride, starches, such as hydroxypropyl starch phosphate, potato starch (modified or unmodified), celluloses such as hydroxyethylcellulose, guars such as hydroxypropyl guar, or mixtures thereof.

In some cases, the thickening agents may include one or more associative thickening polymers such as anionic associative polymers, amphoteric associative polymers, cationic associative polymers, nonionic associative polymers, and mixtures of two or more thereof. A non-limiting example of an amphoteric associative polymer is acrylates/beheneth-25 methacrylate copolymer, sold under the tradename NOVETHIX L-10 (Lubrizol). Non-limiting examples of anionic associative polymers include INCI name: acrylates copolymer, sold under the tradename CARBOPOL Aqua SF-1 (Lubrizol), INCI name: acrylates crosspolymer-4, sold under the tradename CARBOPOL Aqua SF-2 (Lubrizol), and mixtures of two or more thereof. The associative thickening polymers, for instance, the acrylates copolymer and/or the acrylates crosspolymer-4, may be neutralized in water or an aqueous solution with a neutralizing agent before the polymer is added into a hair color base or hair color toning composition.

In some embodiments, the thickener is chosen from hydroxyethylcellulose, cetyl hydroxyethylcellulose, or combinations thereof.

The total amount of the one or more thickening agents may vary, but in some cases is about 0.1% to about 15%, about 0.1% to about 10%, about 0.1% to about 8%, about 0.1% to about 6%, about 0.15 to about 5%, about 0.1% to about 4%, about 0.1% to about 3%, about 0.1% to about 2%, about 0.1% to about 1%, about 0.5% to about 10%, about 0.5% to about 8%, about 0.5% to about 5%, about 0.5% to about 2%, about 1% to about 10%, about 1% to about 8%, about 1% to about 6%, or about 1% to about 5% by weight, relative to the total weight of the composition.

### pH Adjusters

The hair color base compositions according to the disclosure typically have a pH ranging from about 7 to about 10.5, such as from about 7.5 to about 10.5, about 8 to about 10.5, about 8.5 to about 10.5, about 8.5 to 10, about 9 to about 10.5, or about 9 to about 10. For example, the hair color base compositions may have a pH of about 7, about 7.5, about 8, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, 10.0, about 10.1, about 10.2, about 10.3, about 10.4, or about 10.5. The compositions may therefore, optionally contain acid and/or alkali pH adjusters, which are well known in the art in the cosmetic treatment of keratin fibers, such as hair. Such pH adjusters include, but are not limited to, sodium metasilicate, silicate compounds, citric acid, ascorbic acid, and carbonate compounds.

### Additional Components

The hair color base and/or hair color toning compositions according to the disclosure may optionally comprise any auxiliary or additional component suitable for use in cosmetic compositions, and in particular suitable for hair color base or hair color toning compositions. Such components may include, but are not limited to, dyes/pigments to color the composition, fillers, structuring agents, propellants, shine agents, antioxidants or reducing agents, penetrants, sequestrants, fragrances, buffers, dispersants, conditioning agents (e.g. plant extracts, conditioning polymers), ceramides, preserving agents, opacifiers, etc.

The hair color base compositions can be in any suitable form, for example gel, liquid, liquid-gel, cream, etc.

### II. Methods

It has been discovered that hair color toning compositions according to the disclosure surprisingly have improved color persistence, provide desirable hair color shades and tones, and also impart excellent cosmetic properties to the hair such as improved strength, shine, conditioning, feel, detangling, and/or combability. Therefore, another aspect of the disclosure pertains to methods of using any of the compositions described herein to alter the color of the hair, to provide desirable shades and/or tones to the hair, to provide wash-resistant color to the hair, and/or to provide cosmetic benefits to the hair. The methods comprise applying the compositions to the hair.

Typically, the hair color toning compositions will be formed by mixing a hair color base according to the disclosure with an oxidizing composition comprising at least one oxidizing agent (also known as a per composition or a developer) comprising one or more oxidizing agents, and subsequently the hair color toning composition will be applied to the hair. However, it is also contemplated that oxygen in the air may also be employed as an oxidizing agent. In such embodiments, the hair color base composition may not be mixed with a developer composition prior to use.

In embodiments where the hair color base composition is mixed with a developer composition, mix ratios of hair color base composition:developer composition may range from 3:1 to 1:3, such as 2:1 to 1:2, 1.5:1 to 1:1.5, or 1:1. In such embodiments, it will be understood that amounts of components described herein for the hair color base and developer compositions will be correspondingly reduced in the resulting hair color toning composition. The hair color base composition and developer composition are typically mixed at or near the time of use.

In various embodiments the oxidizing agent may be chosen, for example, from peroxides, persulfates, perborates, percarbonates, alkali metal bromates, ferricyanides, peroxygenated salts, alkali metal carbonates, or mixtures of two or more thereof. Oxidizing agents that may also be used include at least one redox enzyme such as laccases, peroxidases, and 2-electron oxidoreductases, such as uricase, where appropriate in the presence of their respective donor or co-factor.

In various embodiments, the oxidizing agent can be hydrogen peroxide present in an aqueous solution whose titer may range from 1 to 40 volumes, such as from 5 to 40 volumes or such as from 5 to 20 volumes.

In another embodiment, the oxidizing agent can be a persulfate and/or a monopersulfate such as, for example, potassium persulfate, sodium persulfate, ammonium persulfate, as well as mixtures thereof. In various embodiments, the oxidizing agents are chosen from hydrogen peroxide, potassium persulfate, sodium persulfate, and mixtures thereof. In certain embodiments, the oxidizing agent is hydrogen peroxide.

In general, the oxidizing agent will be present in an amount ranging from about 0.05 to about 50% by weight, such as from about 0.1% to about 30% by weight, from about 0.1% to about 20% by weight, from about 1% to about 10% by weight, or from about 2% to about 3% based on the total weight of the developer composition or solution or system in which it is present.

In some embodiments, the developer composition is aqueous or is in the form of an emulsion. In such embodiments the developer composition can contain at least one solvent, chosen from water, organic solvents, and mixtures thereof. For example, the developer composition may contain from about 75 to 98% by weight, from about 80 to 98% by weight, from about 85 to 98% by weight, or from about 90 to 98% of water.

In alternative embodiments, the developer composition is substantially anhydrous. The term “substantially anhydrous” means that the developer composition is either completely free of water or contains no appreciable amount of water, for example, no more than 5% by weight, or no more than 2% by weight, or no more than 1% by weight, based on the weight of the developer composition. It should be noted that this refers for example to bound water, such as the water of crystallization of the salts or traces of water absorbed by the raw materials used in the preparation of the compositions according to embodiments of the disclosure.

When the developer composition is substantially anhydrous, the developer composition may comprise at least one solvent chosen from organic solvents. Suitable organic solvents for use in the developer composition include ethanol, isopropyl alcohol, propanol, benzyl alcohol, phenyl ethyl alcohol, glycols and glycol ethers, such as propylene glycol, hexylene glycol, ethylene glycol monomethyl, monoethyl or monobutyl ether, propylene glycol and its ethers, such as propylene glycol monomethyl ether, butylene glycol, dipropylene glycol, diethylene glycol alkyl ethers, such as diethylene glycol monoethyl ether and monobutyl ether, ethylene glycol, propylene glycol, butylene glycol, hexylene glycol, propane diol, glycerin, hydrocarbons such as straight chain hydrocarbons, mineral oil, polybutene, hydrogenated polyisobutene, hydrogenated polydecene, polydecene, squalane, petrolatum, isoparaffins, and mixtures, thereof.

The organic solvents for use in the developer composition can include volatile or non-volatile compounds. The organic solvent may, for example, be present in an amount ranging from about 0.5% to about 70% by weight, such as from about 2% to about 60% by weight, preferably from about 5 to about 50% by weight, relative to the total weight of the developer composition or system in which it is present.

The developer composition may contain additional ingredients such as fatty alcohols, nonionic surfactants, chelants, preservatives, and glycerin may be present.

The developer composition may be in the form of a powder, gel, liquid, foam, lotion, cream, mousse, emulsion, etc.

The pH of the developer composition can range from about 2 to about 12, such as from about 6 to about 11, and it may be adjusted to the desired value using acidifying/alkalizing agents that are well known in the art. In certain embodiments, the pH of the developer composition is below 7.

The pH of the hair color toning composition resulting from mixing together the hair color base composition and the developer composition may range from about 6.0 to about 6.9, such as about 6.0 to about 6.8, about 6.0 to about 6.7, about 6.0 to about 6.6, about 6.0 to about 6.5, about 6.1 to about 6.7, about 6.1 to about 6.6, about 6.1 to about 6.5, about 6.2 to about 6.7, about 6.2 to about 6.6, about 6.2 to about 6.5, about 6.3 to about 6.7, about 6.3 to about 6.6, about 6.3 to about 6.5, about 6.4 to about 6.7, about 6.4 to about 6.6, or about 6.4 to about 6.5. For example, the pH of the mixture may be about 6.0, about 6.1, about 6.2, about 6.3, about 6.4, about 6.5, about 6.6, about 6.7, or about 6.8.

After application to the hair, the hair color base or hair color toning composition may be left to process on the hair for a period of time sufficient to achieve the desired effect. For example, the hair color base or hair color toning composition may be left on the hair for up to one hour, such as from about 3 minutes to about 45 minutes, from about 5 minutes to about 30 minutes, from about 5 minutes to about 25 minutes, or from about 10 minutes to about 20 minutes. In further embodiments, the hair color base or hair color toning composition may be left on the hair for a period up to about 30 minutes, such as, for example, from about 1 to about 30 minutes, about 1 to about 10 minutes, or about 1 to about 5 minutes. For example, the hair color base or hair color toning composition may be left on the hair for about 5 minutes, about 8 minutes, about 10 minutes, about 15 minutes, about 20 minutes, about 25 minutes, or about 30 minutes. One skilled in the art will be able to determine an appropriate amount of time to leave the hair color base or hair color toning composition on the hair in order to achieve the desired coloring effect. If desired, the composition may, optionally, be shampooed and/or rinsed off the hair.

### III. Kits

Another aspect of the disclosure pertains to kits which comprise any of the hair color base compositions described herein. In some embodiments, the kit comprises a hair color base composition according to the disclosure in a first compartment or container, and an oxidizing agent or oxidizing composition in a second compartment or container. The oxidizing composition and the hair color base composition may be mixed prior to application onto hair. Optionally, the kit may comprise at least one additional compartment or container, for example comprising an additional hair treatment composition, and/or may comprise additional implements such as those needed to form the hair color toning composition and/or apply the hair color toning composition to the hair.

In various embodiments, the kits comprising the hair color base compositions according to the disclosure and optionally the developer composition are free or substantially free of ammonia.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense.

The terms “a,” “an,” and “the” are understood to encompass the plural as well as the singular.

The terms “a mixture thereof,” “a combination thereof,” “mixtures thereof,” “mixtures of two or more thereof,” “combinations thereof,” and “combinations of two or more thereof” are used interchangeably herein to mean at least two of the preceding listed elements as shown in the following example where letters A-F represent the elements: “one or more elements chosen from A, B, C, D, E, F, and a mixture thereof.” The terms “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements chosen from A, B, C, D, E, F, and a mixture of any two or more of A, B, C, D, E, and F.”

Likewise, the term “a salt thereof” also relates to “salts thereof.” Thus, where the disclosure refers to “an element chosen from A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be include, or a mixture of any two of A, B, C, D, E, F, a salt of A, a salt of B, a salt of C, a salt of D, a salt of E, and a salt of F may be included.

The salts, for example the salts of the amino acids and the carboxylic acids, which are referred to throughout the disclosure may include salts having a counter-ion such as an alkali metal, alkaline earth metal, or ammonium counterion. This list of counterions, however, is non-limiting.

The expression “one or more” means “at least one” and thus includes individual components as well as mixtures/combinations. For example, “the total amount of the at least one anionic surfactant” refers to the total amount of all anionic surfactants present in the composition.

The term “color” and variations thereof should be understood to refer to the resulting color on the hair at a specified point in time. For example, the color can be the result of dyeing the hair with a hair toning composition formed by mixing a hair color base composition according to the disclosure with an oxidizing composition, or can be the resulting color after a specified number of washes.

The terms “shades,” “tones,” “color tones,” “color reflect,” “hue” and variations thereof should be understood to refer to various shades and tones of the resulting color at a specified time. For example, in the disclosure, a brown color result can include various shades of brown, such as ash brown, mahogany brown, violet brown, copper brown, etc., and any various tone of such brown shades.

The term “color space” and variations thereof should be understood to refer to the color profile based on CIE color mapping as would be understood by those who are skilled in the art of hair color.

The term “similar color” should be understood to mean+/−one level and a color reflect on hair that appears visually similar by the unaided human eye. For example, if a shade is meant to produce a level 6, natural shade, with no additional vibrancy reflect it would be commercially available as a Level 6 Natural shade. If a shade is meant to produce a level 9, with a violet vibrancy reflect (for toning, neutralizing or coloring) it would be commercially available as a “Level 9, Violet” shade. The comparisons made should be understood to be between similar shades. For example, the color retention of “Level 9 Violet” hair color toning composition according to the disclosure would be compared to a commercially available “Level 9 Violet” product that demonstrate a +/−one level difference and a color reflect on hair that appears visually similar by the unaided human eye.

The term “color retention,” “color fade resistance,” “color longevity,” “color persistence,” “wash resistance,” and variations thereof should be understood to refer to the lastingness of the original color imparted onto the hair with the hair color base or hair color toning composition over long term exposure to environmental and mechanical damages, including multiple washes. Differences in color retention (wash resistance) can be determined by comparing the ΔE, ΔL, Δa, and/or Δb of the hair treated with a hair color base or hair color toning composition over multiple washes with that of hair treated with a hair color base or hair color toning composition of a similar color or tone.

Other than in the operating examples, or where otherwise indicated, all numbers expressing quantities of ingredients and/or reaction conditions are to be understood as being modified in all instances by the term “about,” meaning within +/−5% of the indicated number.

All percentages, parts and ratios herein are based upon the total weight of the compositions described, unless otherwise indicated.

“Keratinous substrates” as used herein, includes, but is not limited to keratin fibers such as hair and/or scalp on the human head.

“Conditioning” as used herein means imparting to one or more hair fibers at least one property chosen from combability, moisture-retentivity, luster, shine, and/or softness. The state of conditioning can be evaluated by any means known in the art, such as, for example, measuring, and comparing, the ease of combability of the treated hair and of the untreated hair in terms of combing work (gm-in), and consumer perception.

The term “treat” (and its grammatical variations) as used herein refers to the application of the compositions of the present disclosure onto the surface of keratinous substrates such as hair. The term “treat” (and its grammatical variations) as used herein also refers to contacting keratinous substrates such as hair with the compositions of the present disclosure.

A “rinse off” product refers to a composition such as a hair color toning composition that is rinsed and/or washed with water either after or during the application of the composition onto the keratinous substrate, and before drying and/or styling said keratinous substrate. At least a portion, and typically most, of the composition is removed from the keratinous substrate during the rinsing and/or washing.

The term “stable” as used herein means that the composition does not exhibit change in color, phase separation, and/or crystallization for a period of time, for example, for at least 1 day (24 hours), one week, one month, or one year. The term “stable” can also mean that the color imparted by the hair color base or hair color toning composition does not change over a specified period of time.

“Volatile”, as used herein, means having a flash point of less than about 100° C.

“Non-volatile”, as used herein, means having a flash point of greater than about 100° C.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range.

Unless otherwise specified for a particular component, as used herein the terms “substantially free” or “essentially free” mean the component may be added in amounts less than 2% by weight, based on the total weight of the composition in which it is present, such as less than 1.75%, less than 1.5%, less than 1.25%, less than 1%, less than 0.75%, less than 0.5%, less than 0.25%, less than 0.1%, less than 0.05%, or less than 0.01% of the specified component. Unless expressly stated otherwise, “substantially free” or “essentially free” means less than 2%.

As used herein, if a component is described as being present “in an amount up to” a certain amount, it is intended that such component is, in fact, present in the composition, i.e. is present in an amount greater than 0%.

Furthermore, all components that are positively set forth in the instant disclosure may be negatively excluded from the claims, e.g., a claimed composition may be “free,” “essentially free” (or “substantially free”) of one or more components that are positively set forth in the instant disclosure. The terms “free,” “substantially free,” and “essentially free” as used herein mean that the specific component is not added to the composition but may still be present as an additive in small amounts of a raw material that is included in the composition. For example, a composition that has no added waxes is “free of waxes” even though the composition comprises a pigment that is coated with a wax.

The following examples serve to illustrate embodiments of the present disclosure without, however, being limiting in nature. It will be apparent to those skilled in the art that various modifications and variations can be made in the delivery system, composition and methods of the invention without departing from the spirit or scope of the invention. Thus, it is intended that the present disclosure cover the modifications and variations that come within the scope of the appended claims and their equivalents.

### EXAMPLES

Implementation of the present disclosure is demonstrated by way of the following non-limiting Examples, which are intended to be non-limiting and explanatory in nature only. In the Examples, amounts are expressed in percentage by weight (wt %) of active materials, relative to the total weight of the composition, unless otherwise indicated.

In the Examples, color was measured immediately after the swatches were treated with the hair color toning composition, rinsed, washed with a commercially available shampoo, rinsed, and dried with heat (wash cycle 1=T₁). Additional embodiments were subjected to numerous wash and dry cycles using the same commercially available shampoo for the specified number of wash cycles, for example ten wash cycles (T₁₀), twenty wash cycles (T₂₀), or twenty-eight wash cycles (T₂₈). The color results were measured on a Colorshot MS colorimeter, and color coordinates were recorded using the CIE L*a*b* system.

The L*a*b* data uses numerical values to characterize a specific point in color space and can be used to easily identify minute color differences in a numerical format.

In this system, the three parameters represent, respectively, the color intensity (L*), the green/red color axis (a*) and the blue/yellow color axis (b*). The L*CIE value is always positive and indicates the color lightness. The higher the value of L*, the lighter the color. The a* and b*CIE values represent a directional chromaticity coordinate. A positive a* value is the red axis and negative a* is the green axis, therefore the higher the value of a*, the redder the color. A positive b* is the yellow axis and negative b* is the blue axis, therefore, the higher the value of b*, the yellower the color.

The a* and b* data can also be used together to understand how neutral or vibrant the shade reflect is. According to color theory, red and green neutralize each other and similarly, blue and yellow neutralize each other. Therefore, in cases where the directional chromaticity coordinate is closer to the zero point of the directional chromaticity coordinate the values of a and b axis represent a more balanced amount of the red/green value and the blue/yellow value, which is found in more neutral colors such as brown. A more vibrant color effect such as red or copper can be understood by seeing a very strong positive a* and b* value. A color reflect can represent a more neutral shade such as brown with a slight hint of a red and/or blue color reflect.

Holistically, the change in the L*a*b* color space can be expressed as a single numerical value ΔE, as mathematically defined as:

$$Δ {E}_{ab}^{*} = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}} .$$

When comparing color, frequently ΔE is evaluated. The closer the ΔE value is to zero, the smaller the difference is in the color space. The ΔE value can describe many different situations, for example, it may be necessary to calculate the ΔE between 2 colors to show the similarity or lack of similarity within the color space. In some cases, it is necessary to understand the change in lightness or color along a certain axis, such as the change in color along the blue/yellow color axis (Ab). These are defined as:

$$Δ L = L 2 - L 1$$

$$Δ a = a 2 - a 1$$

$$Δ b = b 2 - b 1.$$

When comparing a feature such as color persistence (or retention of the color or wash resistance) determining the ΔE, as described in the mathematical equation above, can be useful. The L*2, a*2, b*2 numerical values representing the color result after certain number of wash cycles compared with the L*1, a*1, b*1 numerical values representing the color result from the original wash cycle can likewise illustrate the change across the color space.

The same calculation was made for a control. The color retention (wash resistance) was assessed by comparing the ΔE of the test composition over multiple washes to that of a close control over multiple washes. Within this comparison, the composition with the smaller ΔE demonstrates better wash resistance.

### Example 1—Hair Color Toning Compositions

Hair color toning compositions I1-I8 according to the disclosure were prepared by mixing hair color base compositions I1′-I8′ (Tables 1A-1B) with D1 developer composition at a 1:1 mix ratio (Table 1C).

Each of hair color base compositions I1′-I8′ included the components in Table 1A in the amounts shown. The specific types and amounts of dyes present in each of compositions I1′-I8′ are shown in Table 1B. The pH of each of compositions I1′-I8′ were pH adjusted with monoethanolamine measured to be in the range of about 7 to about 10.5. The hair color base compositions were also stable in physical bulk appearance, mix pH, color of the mix, viscosity, and/or color performance as described in Example 6.

TABLE 1A

| COMPONENT | AMOUNT |
|---|---|
| TAURINE | 0.50 |
| CITRIC ACID | 0.20 |
| MONOETHANOLAMINE | 0.35 |
| SURFACTANTS (sodium C14-16 olefin sulfonate, coco- | 16.25 |
| betaine, cocamide mipa) | |
| FATTY COMPOUNDS (cetyl alcohol, deceth-3, lauryl | 19.00 |
| alcohol, myristyl alcohol, oleyl alcohol, PPG-5-ceteth- | |
| 10-phosphate, PEG/PPG-4/12 dimethicone) | |
| ADDITIVES (preservatives, fragrance, fillers, buffers, | ≤6.00 |
| chelators, antioxidants, vegetal extracts) | |
| SOLVENTS (water and non-aqueous solvents) | QS to 100 |

TABLE 1B

| | I1′ | I2′ | I3′ | I4′ | I5′ | I6′ | I7′ | I8′ |
|---|---|---|---|---|---|---|---|---|
| TOLUENE-2,5-DIAMINE | 0.302 | 0.302 | 0.545 | 0.119 | 0.099 | 0.142 | 0.436 | 0.109 |
| HYDROXYETHYL-3,4- | | | | | | | 0.090 | |
| METHYLENEDIOXY | | | | | | | | |
| ANILINE HCL | | | | | | | | |
| 2-AMINO-4-HYDROXY- | 0.7 | | | | | 0.010 | 0.350 | 0.027 |
| ETHYLAMINOANISOLE | | | | | | | | |
| SULFATE | | | | | | | | |
| 1-NAPHTHOL | | 0.361 | 0.700 | 0.108 | 0.100 | 0.010 | | 0.072 |
| 5-AMINO-6-CHLORO-o- | | | 0.020 | | | | | |
| CRESOL | | | | | | | | |
| m-AMINOPHENOL | | | | 0.036 | 0.030 | 0.138 | 0.300 | 0.034 |

Table 1C is an exemplary 6V developer composition D1 and an exemplary 10V developer composition D2 that can be mixed with the hair color base compositions made from Table 1A and Table 1B. They can be mixed in ratios of hair color base composition:developer composition ranging from 3:1 to 1:3, such as 2:1 to 1:2, 1.5:1 to 1:1.5, or 1:1. The developers had a pH of about 2.2.

TABLE 1C
Developer Compositions

| | D1 | D2 |
|---|---|---|
| HYDROGEN PEROXIDE | 2.0 | 3.0 |
| pH ADJUSTER | QS to pH ~2.2 | QS to pH ~2.2 |
| WATER | QS to 100 | QS to 100 |

### Example 2—Comparative Hair Color Toning Compositions

Comparative hair color toning compositions C1-C8 were prepared by mixing hair color base compositions C1′-C8′ (Tables 2A-2B) with D1 developer composition at a 1:1 mix ratio (Table 1C). The combinations of oxidative dyes (bases and couplers) in compositions C1′-C8′ are different from the combinations in the hair color base compositions of Example 1. Comparative hair color toning compositions C1-C8 were used as benchmark controls for the desired color or tone to be deposited and retained on hair by the formulations of Example 1. A comparison of the similarity in color can be found in Example 3. A comparison of the color persistence analysis can be found in Example 4 and 5.

Each of hair color base compositions C1′-C8′ included the components in Table 2A in the amounts shown. The specific types and amounts of dyes present in each of compositions C1′-C8′ are shown in Table 2B. The pH of each of compositions C1′-C8′ were pH adjusted with monoethaolamine and measured to be in the range of about 7 to about 10.5.

TABLE 2A

| COMPONENT | AMOUNT |
|---|---|
| TAURINE | 0.50 |
| CITRIC ACID | 0.20 |
| MONOETHANOLAMINE | 0.35 |
| SURFACTANTS (sodium C14-16 olefin sulfonate, coco- | 16.25 |
| betaine, cocamide mipa) | |
| FATTY COMPOUNDS (cetyl alcohol, deceth-3, lauryl | 19.00 |
| alcohol, myristyl alcohol, oleyl alcohol, PPG-5-ceteth- | |
| 10-phosphate, PEG/PPG-4/12 dimethicone) | |
| ADDITIVES (preservatives, fragrance, fillers, buffers, | ≤6.00 |
| chelates, antioxidants, vegetal extracts) | |
| SOLVENTS (water and non-aqueous solvents) | QS to 100 |

TABLE 2B

| INCI NAME | C1′ | C2′ | C3′ | C4′ | C5′ | C6′ | C7′ | C8′ |
|---|---|---|---|---|---|---|---|---|
| TOLUENE-2,5-DIAMINE | 0.303 | | | 0.198 | 0.064 | 0.082 | 0.297 | 0.070 |
| 2-METHOXYMETHYL-P- | | 0.381 | 0.381 | | | | | |
| PHENYLENEDIAMINE | | | | | | | | |
| p-AMINOPHENOL | | | | | | 0.007 | | |
| 2-AMINO-4- | | 0.7 | | | | | | |
| HYDROXYETHYLAMINO | | | | | | | | |
| ANISOLE SULFATE | | | | | | | | |
| 1-NAPHTHOL | | | 0.361 | | | | | |
| 2,4-DIAMINOPHENOXY | 0.605 | | | 0.063 | 0.016 | 0.005 | 0.150 | 0.024 |
| ETHANOL HCL | | | | | | | | |
| 4-AMINO-2- | | | | 0.145 | 0.037 | | | 0.017 |
| HYDROXYTOLUENE | | | | | | | | |
| m-AMINOPHENOL | | | | 0.050 | 0.018 | 0.080 | 0.100 | 0.053 |
| RESORCINOL | | | | | | | 0.150 | |

### Example 3—Demonstration of Similarity of Color Obtained

A study was conducted to evaluate the similarity of color and tone of hair treated with hair color toning compositions according to the present disclosure (Example 1) against the color and tone of hair treated with comparative hair color toning compositions (Example 2). The hair color toning compositions prepared in Examples 1 and 2 were applied to either damp or dry, previously lightened, hair swatches at a rate of 3 grams of mixture per gram of hair. The hair color toning compositions were allowed to process on the hair for periods ranging from 5-20 minutes, with the processing time adjusted to achieve a similar color tone and level comparable to that of the comparative hair color toning compositions. Following the processing time, the swatches were rinsed, washed once with shampoo, rinsed again, and dried with heat.

Table 3 shows comparison of the similarity of color and shades obtained with hair color toning compositions. Visual evaluation of the color confirmed that each pair appeared similar.

TABLE 3

| | Composition | | | |
|---|---|---|---|---|
| | According to | | Comparative | |
| Pairing | the Disclosure | Level/Tone | Composition | Level/Tone |
| I1/C1 | I1 | 1/Blue | C1 | 1/Blue |
| I1/C2 | I1 | 1/Blue | C1 | 1/Blue |
| I3/C4 | I3 | 8/Violet Blue | C4 | 8/Violet Blue |
| I5/C5 | I5 | 10/Natural | C5 | 9/Violet |
| | | Violet | | |
| I7/C7 | I7 | 6/Blue Brown | C7 | 6/Blue Brown |

### Example 4—Color Persistence Study

Table 4A shows the CIE color measurement data for the comparison of similar level 1, blue hair color shades. The control hair color toning composition C1 and hair color toning composition 11 according to the disclosure were applied to separate swatches of damp hair and processed for 5 minutes. Following the processing time, the swatches were rinsed, washed once with shampoo, rinsed again, and dried with heat. Once dry, the shade similarity analysis was done on the resulting color and it was determined that both shades were visually similar in level and had a visible blue reflect. Subsequently, the swatches were subjected to a 10 wash/rinse cycle as described above. The CIE color measurement data below shows the L*, a*, b*, and ΔE (color change) calculation between the resulting initial color (T₁) and the resulting color after 10 washes (T₁₀).

TABLE 4A

| | | | Processing | | | | | |
|---|---|---|---|---|---|---|---|---|
| Swatch | | Substrate | Time (min) | CIE | L* | a* | b* | ΔE |
| C1 | T₁ | Damp | 5 | L*1, a*1, b*1 | 9.58 | 8.1 | −14.7 | 49.81 |
| | T₁₀ | Damp | 5 | L*2, a*2, b*2 | 53.84 | −1.11 | 6.22 | |
| I1 | T₁ | Damp | 5 | L*1, a*1, b*1 | 11.59 | 10.24 | −19.46 | 41.44 |
| | T₁₀ | Damp | 5 | L*2, a*2, b*2 | 46.79 | 0.65 | 0.19 | |

A ΔE of 49.81 was calculated for the control C1, between the T₁ and T₁₀, washes. A ΔE of 41.44 was calculated for the hair color toning composition according to the disclosure I1, between T₁ and T₁₀.

Table 4B shows the CIE color measurement data for the comparison of similar level 1, blue hair color shades. The control hair color toning composition C1 and the hair color toning composition 11 according to the disclosure were applied to separate swatches of damp hair and processed for 10 minutes. Following the processing time, the swatches were rinsed, washed once with shampoo, rinsed again, and dried with heat. Once dry, the shade similarity analysis was done on the resulting color and it was determined that both shades were visually similar in level and had a visible blue reflect. Subsequently, the swatches were subjected to a 10 wash/rinse cycle as described above. The CIE color measurement data below shows the L*, a*, b*, and ΔE calculation between the initial color (T₁) and the color after 10 washes (T₁₀).

TABLE 4B

| | | | Processing | | | | | |
|---|---|---|---|---|---|---|---|---|
| Swatch | | Substrate | Time (min) | CIE | L* | a* | b* | ΔE |
| C1 | T₁ | Damp | 10 | L*1, a*1, b*1 | 13.54 | 9.16 | −22.72 | 48.61 |
| | T₁₀ | Damp | 10 | L*2, a*2, b*2 | 51.56 | −1.48 | 5.64 | |
| I1 | T₁ | Damp | 10 | L*1, a*1, b*1 | 8.85 | 9.1 | −16.65 | 39.89 |
| | T₁₀ | Damp | 10 | L*2, a*2, b*2 | 44.97 | 0.95 | −1.83 | |

A ΔE of 48.61 was calculated for the control C1, between the T₁ and T₁₀, washes. A ΔE of 39.89 was calculated for the hair color toning composition according to the disclosure I1, between T₁ and T₁₀.

This Example demonstrates that the hair color toning composition according to the disclosure I1 surprisingly had a significantly lower ΔE color change under different processing conditions, indicating it had better color retention (color persistence) performance over the control formula C1.

### Example 5—Tables of Additional Color Persistence Studies

Example 5 shows the CIE color measurement data, as described above, of additional comparisons. The ΔE calculations and color retention (color persistence) evaluations were completed, as described in Example 4.

Table 5A shows the CIE color measurement data for the comparison of similar level 1, blue hair color shades. The control hair color toning composition C2 and the hair color toning composition 11 according to the disclosure were applied to separate swatches of dry hair and processed for 20 minutes. Following the processing time, the swatches were rinsed, washed once with shampoo, rinsed again, and dried with heat. Once dry, the shade similarity analysis was done on the resulting color and it was determined that both shades were visually similar in level and had a visible blue reflect. The CIE color measurement data below shows the L*, a*, b*, and ΔE (color change) calculation between the resulting initial color (T₁) and the resulting color after 10 washes (T₁₀).

TABLE 5A

| | | | Processing | | | | | |
|---|---|---|---|---|---|---|---|---|
| Swatch | | Substrate | Time (min) | CIE | L* | a* | b* | ΔE |
| C2 | T₁ | Dry | 20 | L*1, a*1, b*1 | 6.24 | 3.06 | −4.49 | 36.67 |
| | T₁₀ | Dry | 20 | L*2, a*2, b*2 | 42.88 | 2.9 | −3 | |
| I1 | T₁ | Dry | 20 | L*1, a*1, b*1 | 7.18 | 5.61 | −10.53 | 32.86 |
| | T10 | Dry | 20 | L*2, a*2, b*2 | 39.32 | 1.942 | −4.75 | |

Table 5B shows the CIE color measurement data for the comparison of similar level 8, violet blue shades hair color shades. The control hair color toning composition C4 was applied to a swatch of damp hair and processed for 20 minutes. To achieve a similar shade, hair color toning composition 13 according to the disclosure was applied to damp hair and processed for 10 minutes. Following the processing time, the swatches were rinsed, washed once with shampoo, rinsed again, and dried with heat. Once dry, the shade similarity analysis was done on the resulting color and it was determined that both shades were visually similar in level and had a similar degree of visible violet blue reflect. The CIE color measurement data below shows the L*, a*, b*, and ΔE (color change) calculation between the resulting initial color (T₁) and the resulting color after 10 washes (T₁₀).

TABLE 5B

| | | | Processing | | | | | |
|---|---|---|---|---|---|---|---|---|
| Swatch | | Substrate | Time (min) | CIE | L* | a* | b* | ΔE |
| C4 | T₁ | Damp | 20 | L*1, a*1, b*1 | 28.23 | 11 | 2.15 | 26.86 |
| | T₁₀ | Damp | 20 | L*2, a*2, b*2 | 51.05 | 7.49 | 15.87 | |
| I3 | T₁ | Damp | 10 | L*1, a*1, b*1 | 26.36 | 12.88 | 3.16 | 16.47 |
| | T₁₀ | Damp | 10 | L*2, a*2, b*2 | 41.36 | 10.07 | 9.36 | |

Table 5C shows the CIE color measurement data for the comparison of similar level 8, violet blue hair color shades. The control hair color toning composition C4 was applied to a swatch of dry hair and processed for 20 minutes. To achieve a similar shade, hair color toning composition 13 according to the disclosure was applied to dry hair and processed for 10 minutes. Following the processing time, the swatches were rinsed, washed once with shampoo, rinsed again, and dried with heat. Once dry, the shade similarity analysis was done on the resulting color and it was determined that both shades were visually similar in level and had a similar degree of visible violet blue reflect. The CIE color measurement data below shows the L*, a*, b*, and ΔE (color change) calculation between the resulting initial color (T₁) and the resulting color after 10 washes (T₁₀).

TABLE 5C

| | | | Processing | | | | | |
|---|---|---|---|---|---|---|---|---|
| Swatch | | Substrate | Time (min) | CIE | L* | a* | b* | ΔE |
| C4 | T₁ | Dry | 20 | L*1, a*1, b*1 | 27.26 | 11.23 | 1.65 | 26.41 |
| | T₁₀ | Dry | 20 | L*2, a*2, b*2 | 49.17 | 8.05 | 16.04 | |
| I3 | T₁ | Dry | 10 | L*1, a*1, b*1 | 22.4 | 13.84 | 3.48 | 20.08 |
| | T₁₀ | Dry | 10 | L*2, a*2, b*2 | 41.1 | 10.59 | 10.04 | |

Table 5D shows the CIE color measurement data for the comparison of a level 10 natural violet shade with a Level 9 violet shade. The control hair color toning composition C5 was applied to a swatch of damp hair and processed for 20 minutes. To achieve a similar shade, hair color toning composition 15 according to the disclosure was applied to damp hair and processed for 10 minutes. Following the processing time, the swatches were rinsed, washed once with shampoo, rinsed again, and dried with heat. Once dry, the shade similarity analysis was done on the resulting color and it was determined that both shades were visually similar within 1 level and had a similar visible natural violet reflect. The CIE color measurement data below shows the L*, a*, b*, and ΔE (color change) calculation between the resulting initial color (T₁) and the resulting color after 10 washes (T₁₀).

TABLE 5D

| | | | Processing | | | | | |
|---|---|---|---|---|---|---|---|---|
| Swatch | | Substrate | Time (min) | CIE | L* | a* | b* | ΔE |
| C5 | T₁ | Damp | 20 | L*1, a*1, b*1 | 50.40 | 6.25 | 10.73 | 9.84 |
| | T₁₀ | Damp | 20 | L*2, a*2, b*2 | 57.74 | 6.70 | 17.28 | |
| I5 | T₁ | Damp | 10 | L*1, a*1, b*1 | 57.47 | 5.22 | 21.94 | 1.44 |
| | T₁₀ | Damp | 10 | L*2, a*2, b*2 | 58.77 | 4.85 | 21.46 | |

Table 5E shows the CIE color measurement data for the comparison of a level 10 natural violet shade with a Level 9 violet shade. The control hair color toning composition C5 was applied to a swatch of dry hair and processed for 20 minutes. To achieve a similar shade, hair color toning composition 15 according to the disclosure was applied to dry hair and processed for 10 minutes. Following the processing time, the swatches were rinsed, washed once with shampoo, rinsed again, and dried with heat. Once dry, the shade similarity analysis was done on the resulting color and it was determined that both shades were visually similar within 1 level and had a similar visible natural violet reflect. The CIE color measurement data below shows the L*, a*, b*, and ΔE (color change) calculation between the resulting initial color (T₁) and the resulting color after 10 washes (T₁₀).

TABLE 5E

| | | | Processing | | | | | |
|---|---|---|---|---|---|---|---|---|
| Swatch | | Substrate | Time (min) | CIE | L* | a* | b* | ΔE |
| C5 | T₁ | Dry | 20 | L*1, a*1, b*1 | 49.32 | 7.73 | 11.49 | 9.66 |
| | T₁₀ | Dry | 20 | L*2, a*2, b*2 | 57.20 | 6.75 | 16.99 | |
| I5 | T₁ | Dry | 10 | L*1, a*1, b*1 | 56.00 | 5.28 | 22.10 | 0.57 |
| | T₁₀ | Dry | 10 | L*2, a*2, b*2 | 56.28 | 5.31 | 21.61 | |

Table 5F shows the CIE color measurement data for the comparison of a level 10 natural violet shade and a Level 9 violet shade. The control hair color toning composition C5 was applied to a swatch of damp hair and processed for 20 minutes. To achieve a similar shade, hair color toning composition 15 according to the disclosure was applied to damp hair and processed for 10 minutes. Following the processing time, the swatches were rinsed, washed once with shampoo, rinsed again, and dried with heat. Once dry, the shade similarity analysis was done on the resulting color and it was determined that both shades were visually similar within 1 level and had a similar visible natural violet reflect. The CIE color measurement data below shows the L*, a*, b*, and ΔE (color change) calculation between the resulting initial color (T₁) and the resulting color after 20 washes (T₂₀).

TABLE 5F

| | | | Processing | | | | | |
|---|---|---|---|---|---|---|---|---|
| Swatch | | Substrate | Time (min) | CIE | L* | a* | b* | ΔE |
| C5 | T₁ | Damp | 20 | L*1, a*1, b*1 | 50.40 | 6.25 | 10.73 | 11.32 |
| | T₂₀ | Damp | 20 | L*2, a*2, b*2 | 59.01 | 7.08 | 18.04 | |
| I5 | T₁ | Damp | 10 | L*1, a*1, b*1 | 57.47 | 5.22 | 21.94 | 1.26 |
| | T₂₀ | Damp | 10 | L*2, a*2, b*2 | 58.71 | 5.05 | 21.99 | |

Table 5G shows the CIE color measurement data for the comparison of a level 10 natural violet shade with a Level 9 violet shade. The control hair color toning composition C5 was applied to a swatch of dry hair and processed for 20 minutes. To achieve a similar shade, hair color toning composition 15 according to the disclosure was applied to dry hair and processed for 10 minutes. Following the processing time, the swatches were rinsed, washed once with shampoo, rinsed again, and dried with heat. Once dry, the shade similarity analysis was done on the resulting color and it was determined that both shades were visually similar within 1 level and had a similar visible natural violet reflect. The CIE color measurement data below shows the L*, a*, b*, and ΔE (color change) calculation between the resulting initial color (T₁) and the resulting color after 20 washes (T₂₀).

TABLE 5G

| | | | Processing | | | | | |
|---|---|---|---|---|---|---|---|---|
| Swatch | | Substrate | Time (min) | CIE | L* | a* | b* | ΔE |
| C5 | T₁ | Dry | 20 | L*1, a*1, b*1 | 49.32 | 7.73 | 11.49 | 12.39 |
| | T₂₀ | Dry | 20 | L*2, a*2, b*2 | 59.16 | 6.92 | 18.98 | |
| I5 | T₁ | Dry | 10 | L*1, a*1, b*1 | 56.00 | 5.28 | 22.10 | 1.46 |
| | T₂₀ | Dry | 10 | L*2, a*2, b*2 | 57.34 | 5.67 | 21.67 | |

Table 5H shows the CIE color measurement data for the comparison of a level 10 natural violet shade with a Level 9 violet shade. The control hair color toning composition C5 was applied to a swatch of damp hair and processed for 20 minutes. To achieve a similar shade, hair color toning composition 15 according to the disclosure was applied to damp hair and processed for 10 minutes. Following the processing time, the swatches were rinsed, washed once with shampoo, rinsed again, and dried with heat. Once dry, the shade similarity analysis was done on the resulting color and it was determined that both shades were visually similar within 1 level and had a similar visible natural violet reflect. The CIE color measurement data below shows the L*, a*, b*, and ΔE (color change) calculation between the resulting initial color (T₁) and the resulting color after 28 washes (T₂₈).

TABLE 5H

| | | | Processing | | | | | |
|---|---|---|---|---|---|---|---|---|
| Swatch | | Substrate | Time (min) | CIE | L* | a* | b* | ΔE |
| C5 | T₁ | Damp | 20 | L*1, a*1, b*1 | 50.40 | 6.25 | 10.73 | 13.02 |
| | T₂₈ | Damp | 20 | L*2, a*2, b*2 | 60.08 | 7.15 | 19.39 | |
| I5 | T₁ | Damp | 10 | L*1, a*1, b*1 | 57.47 | 5.22 | 21.94 | 0.57 |
| | T₂₈ | Damp | 10 | L*2, a*2, b*2 | 57.96 | 5.16 | 21.67 | |

Table 51 shows the CIE color measurement data for the comparison of a level 10 natural violet shade with a Level 9 violet shade. The control hair color toning composition C5 was applied to a swatch of dry hair and processed for 20 minutes. To achieve a similar shade, hair color toning composition 15 according to the disclosure was applied to dry hair and processed for 10 minutes. Following the processing time, the swatches were rinsed, washed once with shampoo, rinsed again, and dried with heat. Once dry, the shade similarity analysis was done on the resulting color and it was determined that both shades were visually similar within 1 level and had a similar visible natural violet reflect. The CIE color measurement data below shows the L*, a*, b*, and ΔE (color change) calculation between the resulting initial color (T₁) and the resulting color after 28 washes (T₂₈).

TABLE 5I

| | | | Processing | | | | | |
|---|---|---|---|---|---|---|---|---|
| Swatch | | Substrate | Time (min) | CIE | L* | a* | b* | ΔE |
| C5 | T₁ | Dry | 20 | L*1, a*1, b*1 | 49.32 | 7.73 | 11.49 | 13.30 |
| | T₂₈ | Dry | 20 | L*2, a*2, b*2 | 60.14 | 6.67 | 19.16 | |
| I5 | T₁ | Dry | 10 | L*1, a*1, b*1 | 56.00 | 5.28 | 22.10 | 1.55 |
| | T₂₈ | Dry | 10 | L*2, a*2, b*2 | 57.46 | 5.81 | 22.24 | |

Table 5J shows the CIE color measurement data for the comparison of similar level 6 blue brown hair color shades. The control hair color toning composition C7 was applied to a swatch of damp hair and processed for 20 minutes. To achieve a similar shade, hair color toning composition 17 according to the disclosure was applied to damp hair and processed for 10 minutes. Following the processing time, the swatches were rinsed, washed once with shampoo, rinsed again, and dried with heat. Once dry, the shade similarity analysis was done on the resulting color and it was determined that both shades were visually similar and had a similar visible blue brown reflect. The CIE color measurement data below shows the L*, a*, b*, and ΔE (color change) calculation between the resulting initial color (T₁) and the resulting color after 10 washes (T₁₀).

TABLE 5J

| | | | Processing | | | | | |
|---|---|---|---|---|---|---|---|---|
| Swatch | | Substrate | Time (min) | CIE | L* | a* | b* | ΔE |
| C7 | T₁ | Damp | 20 | L*1, a*1, b*1 | 9.40 | 2.39 | −1.27 | 18.62 |
| | T₁₀ | Damp | 20 | L*2, a*2, b*2 | 23.01 | 6.32 | 10.82 | |
| I7 | T₁ | Damp | 10 | L*1, a*1, b*1 | 11.17 | 4.90 | −7.39 | 6.92 |
| | T₁₀ | Damp | 10 | L*2, a*2, b*2 | 17.54 | 5.89 | −4.87 | |

Table 5K shows the CIE color measurement data for the comparison of similar level 6 blue brown hair color shades. The control hair color toning composition C7 was applied to a swatch of dry hair and processed for 20 minutes. To achieve a similar shade, hair color toning composition 17 according to the disclosure was applied to dry hair and processed for 10 minutes. Following the processing time, the swatches were rinsed, washed once with shampoo, rinsed again, and dried with heat. Once dry, the shade similarity analysis was done on the resulting color and it was determined that both shades were visually similar and had a similar visible blue brown reflect. The CIE color measurement data below shows the L*, a*, b*, and ΔE (color change) calculation between the resulting initial color (T₁) and the resulting color after 10 washes (T₁₀).

TABLE 5K

| | | | Processing | | | | | |
|---|---|---|---|---|---|---|---|---|
| Swatch | | Substrate | Time (min) | CIE | L* | a* | b* | ΔE |
| C7 | T₁ | Dry | 20 | L*1, a*1, b*1 | 8.16 | 1.87 | −0.48 | 23.02 |
| | T₁₀ | Dry | 20 | L*2, a*2, b*2 | 27.96 | 4.57 | 10.95 | |
| I7 | T₁ | Dry | 10 | L*1, a*1, b*1 | 9.48 | 4.29 | −7.02 | 9.96 |
| | T₁₀ | Dry | 10 | L*2, a*2, b*2 | 19.16 | 5.46 | −5.03 | |

Table 5L shows the CIE color measurement data for the comparison of similar level 6 blue brown hair color shades. The control hair color toning composition C7 was applied to a swatch of damp hair and processed for 20 minutes. To achieve a similar shade, hair color toning composition 17 according to the disclosure was applied to damp hair and processed for 10 minutes. Following the processing time, the swatches were rinsed, washed once with shampoo, rinsed again, and dried with heat. Once dry, the shade similarity analysis was done on the resulting color and it was determined that both shades were visually similar and had a similar visible blue brown reflect. The CIE color measurement data below shows the L*, a*, b*, and ΔE (color change) calculation between the resulting initial color (T₁) and the resulting color after 20 washes (T₂₀).

TABLE 5L

| | | | Processing | | | | | |
|---|---|---|---|---|---|---|---|---|
| Swatch | | Substrate | Time (min) | CIE | L* | a* | b* | ΔE |
| C7 | T₁ | Damp | 20 | L*1, a*1, b*1 | 9.40 | 2.39 | −1.27 | 29.09 |
| | T₂₀ | Damp | 20 | L*2, a*2, b*2 | 33.11 | 6.49 | 15.09 | |
| I7 | T₁ | Damp | 10 | L*1, a*1, b*1 | 11.17 | 4.90 | −7.39 | 12.23 |
| | T₂₀ | Damp | 10 | L*2, a*2, b*2 | 21.67 | 5.76 | −1.19 | |

Table 5M shows the CIE color measurement data for the comparison of similar level 6 blue brown hair color shades. The control hair color toning composition C7 was applied to a swatch of dry hair and processed for 20 minutes. To achieve a similar shade, hair color toning composition 17 according to the disclosure was applied to dry hair and processed for 10 minutes. Following the processing time, the swatches were rinsed, washed once with shampoo, rinsed again, and dried with heat. Once dry, the shade similarity analysis was done on the resulting color and it was determined that both shades were visually similar and had a similar visible blue brown reflect. The CIE color measurement data below shows the L*, a*, b*, and ΔE (color change) calculation between the resulting initial color (T₁) and the resulting color after 20 washes (T₂₀).

TABLE 5M

| | | | Processing | | | | | |
|---|---|---|---|---|---|---|---|---|
| Swatch | | Substrate | Time (min) | CIE | L* | a* | b* | ΔE |
| C7 | T₁ | Dry | 20 | L*1, a*1, b*1 | 8.16 | 1.87 | −0.48 | 34.54 |
| | T₂₀ | Dry | 20 | L*2, a*2, b*2 | 36.40 | 7.65 | 18.56 | |
| I7 | T₁ | Dry | 10 | L*1, a*1, b*1 | 9.48 | 4.29 | −7.02 | 11.9 |
| | T₂₀ | Dry | 10 | L*2, a*2, b*2 | 20.26 | 5.67 | −2.18 | |

Table 5N shows the CIE color measurement data for the comparison of similar level 6 blue brown hair color shades. The control hair color toning composition C7 was applied to a swatch of damp hair and processed for 20 minutes. To achieve a similar shade, hair color toning composition 17 according to the disclosure was applied to damp hair and processed for 10 minutes. Following the processing time, the swatches were rinsed, washed once with shampoo, rinsed again, and dried with heat. Once dry, the shade similarity analysis was done on the resulting color and it was determined that both shades were visually similar and had a similar visible blue brown reflect. The CIE color measurement data below shows the L*, a*, b*, and ΔE (color change) calculation between the resulting initial color (T₁) and the resulting color after 28 washes (T₂₈).

TABLE 5N

| | | | Processing | | | | | |
|---|---|---|---|---|---|---|---|---|
| Swatch | | Substrate | Time (min) | CIE | L* | a* | b* | AE |
| C7 | T₁ | Damp | 20 | L*1, a*1, b*1 | 9.40 | 2.39 | −1.27 | 33.43 |
| | T₂₈ | Damp | 20 | L*2, a*2, b*2 | 35.71 | 7.65 | 18.67 | |
| I7 | T₁ | Damp | 10 | L*1, a*1, b*1 | 11.17 | 4.90 | −7.39 | 23.79 |
| | T₂₈ | Damp | 10 | L*2, a*2, b*2 | 32.14 | 5.77 | 3.80 | |

Table 5O shows the CIE color measurement data for the comparison of similar level 6 blue brown hair color shades. The control hair color toning composition C7 was applied to a swatch of dry hair and processed for 20 minutes. To achieve a similar shade, hair color toning composition 17 according to the disclosure was applied to dry hair and processed for 10 minutes. Following the processing time, the swatches were rinsed, washed once with shampoo, rinsed again, and dried with heat. Once dry, the shade similarity analysis was done on the resulting color and it was determined that both shades were visually similar and had a similar visible blue brown reflect. The CIE color measurement data below shows the L*, a*, b*, and ΔE (color change) calculation between the resulting initial color (T₁) and the resulting color after 28 washes (T₂₈).

TABLE 5O

| | | | Processing | | | | | |
|---|---|---|---|---|---|---|---|---|
| Swatch | | Substrate | Time (min) | CIE | L* | a* | b* | ΔE |
| C7 | T₁ | Dry | 20 | L*1, a*1, b*1 | 8.16 | 1.87 | −0.48 | 34.54 |
| | T₂₈ | Dry | 20 | L*2, a*2, b*2 | 36.40 | 7.65 | 18.56 | |
| I7 | T₁ | Dry | 10 | L*1, a*1, b*1 | 9.48 | 4.29 | −7.02 | 22.67 |
| | T₂₈ | Dry | 10 | L*2, a*2, b*2 | 28.67 | 6.51 | 4.84 | |

The results of the comparisons outlined in Example 5 demonstrate the same trend that is outlined in extensive detail in Example 4. The resulting conclusion is that the compositions according to the disclosure show a surprising and consistently unexpected improvement in the color persistence of the color on the hair after multiple washes, demonstrated by the smaller overall color change (lower ΔE). This is the trend regardless of the processing conditions used.

### Example 6—Stability Testing of Hair Color Base Compositions

Hair color base compositions according to the disclosure and comparative hair base compositions were prepared. Each of the compositions included the components in Table 6A in the amounts shown. The specific types and amounts of dyes present in each of compositions 19′-110′ and C9′-C10′ are shown in Table 6B. The hair base compositions were pH adjusted with monoethanolamine and measured to be in the pH range of about 7 to about 10.5.

TABLE 6A

| COMPONENT | AMOUNT |
|---|---|
| TAURINE | 0.50 |
| CITRIC ACID | 0.20 |
| MONOETHANOLAMINE | 0.35 |
| SURFACTANTS (sodium c14-16 olefin sulfonate, coco- | 16.25 |
| betaine, cocamide MIPA) | |
| FATTY COMPOUNDS (cetyl alcohol, deceth-3, lauryl | 19.00 |
| alcohol, myristyl alcohol, oleyl alcohol, PPG-5-ceteth- | |
| 10-phosphate, PEG/PPG-4/12 dimethicone) | |
| ADDITIVES (preservatives, fragrance, fillers, buffers, | ≤6.00 |
| chelates, antioxidants, vegetal extracts) | |
| SOLVENTS (water and non-aqueous solvents) | QS to 100 |

TABLE 6B

| INCI NAME | I9′ | I10′ | C9′ | C10′ |
|---|---|---|---|---|
| TOLUENE-2,5-DIAMINE | 2.000 | 0.750 | 0.065 | |
| 1-NAPHTHOL | | 0.300 | | |
| 2-AMINO-4- | 0.900 | 0.300 | | |
| HYDROXYETHYLAMINOANISOLE | | | | |
| SULFATE | | | | |
| HYDROXYBENZOMORPHOLINE | 0.945 | | | |
| 2,4-DIAMINOPHENOXY ETHANOL | | | 0.016 | |
| HCL | | | | |
| 2-METHYLRESORCINOL | | | | 0.140 |
| 4-AMINO-2-HYDROXYTOLUENE | | | 0.037 | 0.140 |
| m-AMINOPHENOL | 0.700 | 0.300 | 0.018 | |
| 5-AMINO-6-CHLORO-o-CRESOL | | 0.200 | | |
| HYDROXYETHOXY AMINOPYRAZOLO | | | | 0.500 |
| PYRIDINE HCL | | | | |
| HYDROXYETHYL-3,4- | 0.800 | | | |
| METHYLENEDIOXYANILINE HCL | | | | |

TABLE 6C

| Stability | | I9′ | I9′ | I9′ | I10′ | I10′ | 110′ |
|---|---|---|---|---|---|---|---|
| Attribute | Temp | Standard | 4 week | 8 week | Standard | 4 week | 8 week |
| Bulk homo- | 4° C. | — | conforms | — | — | conforms | — |
| geneity | | | to | | | to | |
| | | | standard | | | standard | |
| | 25° C. | — | — | conforms | conforms to | conforms | — |
| | | | | to | standard | to | |
| | | | | standard | | standard | |
| | 37° C. | — | conforms | — | — | — | — |
| | | | to | | | | |
| | | | standard | | | | |
| | 45° C. | — | conforms | conforms | — | conforms | conforms |
| | | | to | to | | to | to |
| | | | standard | standard | | standard | standard |
| Bulk color | 4° C. | — | conforms | — | — | translucent | — |
| | | | to | | | dark | |
| | | | standard | | | yellow | |
| | 25° C. | — | — | conforms | translucent | — | — |
| | | | | to | dark yellow | | |
| | | | | standard | | | |
| | 37° C. | — | conforms | — | — | translucent | — |
| | | | to | | | dark | |
| | | | standard | | | yellow | |
| | 45° C. | — | conforms | conforms | — | translucent | translucent |
| | | | to | to | | dark | dark |
| | | | standard | standard | | yellow | yellow |
| Color of the | 4° C. | — | conforms | — | — | Light | — |
| hair color | | | to | | | Yellow | |
| toning | | | standard | | | | |
| composition | 25° C. | — | conforms | — | Light | Light | — |
| | | | to | | Yellow | Yellow | |
| | | | standard | | | | |
| | 37° C. | — | — | conforms | — | — | Light |
| | | | | to | | | Yellow |
| | | | | standard | | | |
| | 45° C. | — | conforms | — | — | Light | — |
| | | | to | | | Yellow- | |
| | | | standard | | | Slightly | |
| | | | | | | more | |
| Swatch | 4° C. | — | reflect | — | — | conforms | — |
| color result | | | slightly | | | to | |
| | | | more blue | | | standard | |
| | 25° C. | — | conforms | conforms | — | — | — |
| | | | to | to | | | |
| | | | standard | standard | | | |
| | 37° C. | — | — | — | — | conforms | — |
| | | | | | | to | |
| | | | | | | standard | |
| | 45° C. | — | conforms | conforms | — | conforms | reflect |
| | | | to | to | | to | conforms - |
| | | | standard | standard | | standard | slightly |
| | | | | | | | lighter |

TABLE 6D

| Stability | | C9 | C9 | C9 | C10 | C10 |
|---|---|---|---|---|---|---|
| Attribute | Temp | Standard | 4 week | 8 week | Standard | 4 week |
| Bulk homo- | 4° C. | — | conforms | — | — | precipitation/ |
| geneity | | | to standard | | | crystallization |
| | 25° C. | conforms | conforms | conforms | conforms | precipitation/ |
| | | to standard | to standard | to standard | to standard | crystallization |
| | 37° C. | — | — | conforms | — | — |
| | | | | to standard | | |
| | 45° C. | — | conforms | conforms | — | precipitation/ |
| | | | to standard | to standard | | crystallization |
| Bulk color | 4° C. | — | translucent | — | — | — |
| | | | yellow | | | |
| | 25° C. | translucent | translucent | translucent | — | — |
| | | yellow | yellow | yellow | | |
| | 37° C. | — | — | translucent | — | — |
| | | | | yellow | | |
| | 45° C. | — | translucent | translucent | — | — |
| | | | yellow | yellow | | |
| Color of the | 4° C. | — | blue/lavend | | — | — |
| hair color | | | er | | | |
| toning | 25° C. | light blue | light blue | light green | — | — |
| composition | 37° C. | — | — | light green/ | — | — |
| | | | | yelllow | | |
| | 45° C. | — | light blue | light yellow | — | — |
| Swatch | 4° C. | — | conforms | — | — | — |
| color result | | | to standard | | | |
| | 25° C. | — | — | more violet | — | — |
| | | | | than | | |
| | | | | standard | | |
| | 37° C. | — | — | slightly less | — | — |
| | | | | violet than | | |
| | | | | standard | | |
| | 45° C. | — | reflect | less blue | — | — |
| | | | conforms - | than | | |
| | | | slightly | standard | | |
| | | | lighter | | | |

The formulations were evaluated for stability after being stored at various temperatures (room temperature, 4, 37, and 45° C.) over a period of 4 weeks and 8 weeks. Stability was evaluated by assessing the pH, viscosity, physical appearance of the bulk, homogeneity of the bulk, color performance on swatch of the color level and tone initially (standard) and at each time and temperature point indicated in Tables 6C and 6D.

Hair color base compositions 19′ and 110′ were determined to be stable. The viscosity measurements were in the expected range of about 245 cP to about 567 cP, measured using a Rheomat with an M2 spindle at about 25° C., for a time period of about 30 seconds. The pH measurements of the hair color base compositions were found to be in range of about 7 to about 10.5. The bulk physical appearance was a translucent homogeneous liquid.

At each time point indicated (initial, 4 week, 8 week), hair color base compositions 19′ and 110′ were mixed with developer composition D1 at a 1:1 mix ratio to form hair color toning compositions 19 and 110. The color and pH of the hair color toning compositions were evaluated at each timepoint and was found to conform to the standard. The pH of the hair color toning composition was found to be in range of 6 to 6.8. Finally, hair color toning compositions 19 and 110 were applied to swatches of hair and processed as described above. The swatch color results were found to conform to the standard.

At each time point indicated (initial, 4 week, 8 week), hair color base compositions C9′ and C10′ were mixed with developer composition D1 at a 1:1 mix ratio to form hair color toning compositions C9 and C10. Hair color base composition C9′ was determined to be unstable. While the viscosity, pH measurements of the colorant, pH of the hair color toning composition, bulk physical appearance, and homogeneity were all found to conform to the standard, the color of the hair color toning composition shifted at 4 weeks from light blue at room temperature to a blue lavender, and at 8 weeks shifted to a light green at room temperature, transitioning from light green to light yellow at 37° C. and light yellow at 45° C. This is a visual indicator that the dye composition is not stable in the system. Additionally, the swatch color showed reflect differences. The 8 week, room temperature sample swatch color was more violet than the standard, the 8 week, 37° C. sample swatch color was less violet than the standard, and the 8 week, 45° C. sample swatch color was less blue than the standard.

Hair color base composition C10′ was determined to be unstable. It was observed that precipitation and crystallization of the dyes had occurred and separated from the remaining bulk. No other stability attributes were measured.

The results of this study demonstrate that hair color base and hair color toning compositions according to the disclosure are more stable than compositions having combinations of oxidative dyes not according to the disclosure.

### Example 7—Additional Hair Color Toning Compositions

Hair color base compositions 110′-117′ can be prepared using base 1 or base 2 compositions in Table 7A with the dye combinations in Table 7B, and are likewise expected to be stable and provide similar fade-resistant hair color toning benefits.

TABLE 7A

| | BASE | BASE |
|---|---|---|
| COMPONENT | 1 | 2 |
| AMINO ACID (glycine) | 0.1 | |
| AMINO ACID (taurine) | | 1 |
| CARBOXYLIC ACID (lactic acid) | 0.1 | 1 |
| CARBOXYLIC ACID (malic acid) | | 1 |
| ALKALIZER (organic and/or mineral) | 5 | 11 |
| FATTY COMPOUNDS (e.g. glycol distearate, oleic | 5 | 25 |
| acid, cetyl alcohol, oleyl alcohol, silicones) | | |
| LAURETH-12 AND OLETH-30 | 7.00 | 7.00 |
| THICKENING AGENTS (hydroxyethylcellulose, cetyl | 0.01 | 1.5 |
| hydroxyethylcellulose) | | |
| OPTIONAL INGREDIENTS (e.g. conditioning | ≤2.000 | ≤2.000 |
| polymers) | | |
| ADDITIVES (preservatives, fragrance, fillers, | ≤6.000 | ≤6.000 |
| buffers, chelates, antioxidants, vegetal extracts) | | |
| WATER | QS | QS |

TABLE 7B

| | I10' | I11' | I12' | I13' | I14' | I15' | I16' | I17' |
|---|---|---|---|---|---|---|---|---|
| TOLUENE-2,5- | 0.302 | 0.302 | 0.545 | 0.119 | 0.099 | 0.142 | 0.436 | 0.109 |
| DIAMINE | | | | | | | | |
| HYDROXY | | | | | | | 0.090 | |
| ETHYL-3,4- | | | | | | | | |
| METHYLENEDI- | | | | | | | | |
| OXYANILINE HCL | | | | | | | | |
| 2-AMINO-4- | 0.7 | | | | | 0.010 | 0.350 | 0.027 |
| HYDROXYETHYLA | | | | | | | | |
| MINOANISOLE | | | | | | | | |
| SULFATE | | | | | | | | |
| 1-NAPHTHOL | | 0.361 | 0.700 | 0.108 | 0.100 | 0.010 | | 0.072 |
| 5-AMINO-6- | | | 0.020 | | | | | |
| CHLORO-o-CRESOL | | | | | | | | |
| m-AMINOPHENOL | | | | 0.036 | 0.030 | 0.138 | 0.300 | 0.034 |

Hair color base compositions I10′-I17′, to which any combination of additional hair color agents as described herein can be added to achieve various different shades if desired, can be mixed with an oxidizing composition at or near the time of use as described above or as shown in Table 7C below to form hair color toning compositions according to the disclosure. For example, one or more oxidative dye compounds such as toluene-2,5-diamine as an oxidation base, and at least one coupler chosen from 1-napthol, 2-amino-4-hydroxyethylaminoanisole sulfate, and derivatives thereof, addition salts thereof, solvates thereof, or solvates of salts thereof, or mixtures thereof. Additional hair color agents such as m-aminophenol, p-aminophenol, 2-methyl-5-hydroxyethylaminophenol, 2,4-diaminophenoxyethanol hydrochloride, 4-amino-2-hydroxytoluene, 6-hydroxyindole, etc., and/or one or more direct dyes such as HG Red No. 3, Basic Red 5, Basic Yellow 87, Basic Orange 31, 2-Amino-6-Chloro-4-Nitrophenol, 3-Nitro-p-Hydroxyethylaminophenol, 2-Nitro-5-Glyceryl, etc., can be included as described above.

Table 7C shows an exemplary 20V developer composition (D3) and an exemplary 10V developer composition (D4) that can be mixed with hair color base compositions according to the disclosure, for example I10′-I17′, to prepare hair color toning compositions according to the disclosure. For example, D3 or D4 may be mixed with the hair color base compositions in mix ratios of hair color base composition:developer composition ranging from 3:1 to 1:3, such as 2:1 to 1:2, 1.5:1 to 1:1.5, or 1:1.

TABLE 7C
Developer Compositions

| | D3 | D4 |
|---|---|---|
| TRIDECETH-2 CARBOXAMIDE MEA | 0.85 | 0.82 |
| TETRASODIUM ETIDRONATE | 0.06 | 0.06 |
| SODIUM SALICYLATE | 0.035 | 0.035 |
| GLYCERIN | 0.50 | 0.50 |
| CETEARYL ALCOHOL | 2.28 | 2.28 |
| CETEARETH-25 | 0.57 | 0.57 |
| HYDROGEN PEROXIDE | 6.0 | 3.0 |
| TETRASODIUM PYROPHOSPHATE | 0.04 | 0.04 |
| WATER | QS to 100 | QS to 100 |

The above Examples show that hair color base compositions according to the disclosure are surprisingly stable, and that hair color base and hair color toning compositions according to the disclosure surprisingly have improved color persistence while providing desirable hair color shades and tones, imparting excellent cosmetic properties to the hair such as improved strength, shine, conditioning, feel, detangling, and/or combability.

1. A hair color base composition comprising:
(a) at least one amino acid;
(b) at least one carboxylic acid;
(c) monoethanolamine;
(d) a surfactant mixture comprising (i) at least one amphoteric surfactant and (ii) at least one surfactant chosen from anionic and non-ionic surfactants;
(e) at least one fatty compound;
(f) at least one oxidation base chosen from toluene-2,5-diamine, acid salts thereof, or solvates thereof;
(g) a coupler system comprising at least one coupler chosen from:
(I) 1-napthol, and derivatives thereof, addition salts thereof, solvates thereof, or solvates of salts thereof;
(II) 2-amino-4-hydroxyethylaminoanisole sulfate, and derivatives thereof, addition salts thereof, solvates thereof, or solvates of salts thereof; or
(III) mixtures thereof, and
(h) at least one solvent,
wherein the composition has a pH ranging from about 7 to about 10.5.

2. The hair color base composition according to claim 1, wherein the at least one amino acid is chosen from taurine, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine, or combinations thereof.

3. (canceled)

4. The hair color base composition according to claim 1, wherein the at least one amino acid is present in the composition in an amount ranging from about 0.05% to about 5% by weight, relative to the total weight of the composition.

5. (canceled)

6. The hair color base composition according to claim 1, wherein the at least one carboxylic acid is chosen from oxalic acid, malonic acid, glutaric acid, succinic acid, adipic acid, glycolic acid, citric acid, tartaric acid, malic acid, sebacic acid, maleic acid, fumaric acid, benzoic acid, citraconic acid, aconitic acid, propane-1,2,3-tricarboxylic acid, trimesic acid, or combinations thereof.

7. The hair color base composition according to claim 1, wherein the at least one carboxylic acid is chosen from citric acid.

8. The hair color base composition according to claim 1, wherein the at least one carboxylic acid is present in the composition in an amount ranging from about 0.01% to about 3% by weight, relative to the weight of the composition.

9. (canceled)

10. (canceled)

11. The hair color base composition according to claim 1, wherein the at least one fatty compound is chosen from C12-C22 fatty alcohols.

12. The hair color base composition according to claim 1, wherein the at least one oxidation base is present in the composition in an amount ranging from about 0.001% to about 20% by weight, relative to the total weight of the composition.

13. The hair color base composition according to claim 1, wherein the at least one coupler is present in the composition in an amount ranging from about 0.001% to about 20% by weight, relative to the total weight of the composition.

14. The hair color base composition according to claim 1, further comprising:
(i) one or more additional oxidative bases chosen from p-aminophenol, N,N-bis((2-hydroxyethyl)-p-phenylenediamine sulfate, Hydroxypropyl bis(N-hydroxyethyl-p-phenylenediamine) HCl, O-Aminophenol, N-methyl-p-aminophenol sulfate, N-methyl-p-aminophenol sulfate, 2,3-Diaminohydropyrazolo pyrazolone dimethosulfate, 1-Hydroxyethyl 4,5-diamino pyrazole sulfate, 2-[(3-1-Aminopyrazolo[1,5-A]pyridin-2-yl)oxy]ethanol hydrochloride and their derivatives thereof, their addition salts thereof, their solvates thereof, or their solvates of salts thereof, and mixtures thereof, and/or
(ii) one or more additional couplers chosen from hydroxybenzomorpholine (hydroxy-6-benzomorpholine), 2-amino-3-hydroxypyridine, m-aminophenol, 2-methyl-5-hydroxyethylaminophenol, hydroxy-6-indole, 2-amino-3-hydroxypyridine, 5-amino-6-chloro-o-cresol, 1-naphthol and their derivatives thereof, their addition salts thereof, their solvates thereof, or their solvates of salts thereof, and mixtures thereof.

15. The hair color base composition according to claim 1, further comprising at least one conditioning agent.

16. A method for altering the tone or color of the hair, comprising mixing:
(i) a hair color base composition comprising:
(a) at least one amino acid,
(b) at least one carboxylic acid,
(c) monoethanolamine,
(d) a surfactant mixture comprising (i) at least one amphoteric surfactant and (ii) at least one surfactant chosen from anionic and non-ionic surfactants,
(e) at least one fatty compound,
(f) at least one oxidation base chosen from toluene-2,5-diamine, acid salts thereof, or solvates thereof;
(g) a coupler system comprising at least one coupler chosen from:
(I) 1-napthol, and derivatives thereof, addition salts thereof, solvates thereof, or solvates of salts thereof;
(II) 2-amino-4-hydroxyethylaminoanisole sulfate, and derivatives thereof, addition salts thereof, solvates thereof, or solvates of salts thereof; and
(III) mixtures thereof, and
(h) at least one solvent;
wherein the hair color base composition has a pH ranging from about 7 to about 10.5, with
(ii) an oxidizing composition comprising at least one oxidizing agent to form a hair color toning composition, and applying the hair color toning composition to the hair,
wherein the hair color toning composition has a pH ranging from about 6.0 to about 6.8.

17. The method according to claim 16, wherein the at least one amino acid is chosen from taurine, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine, or combinations thereof.

18. The method according to claim 16, wherein the at least one amino acid is chosen from taurine, glycine, or combinations thereof.

19. The method according to claim 16, wherein the at least one amino acid is present in the hair color base composition in an amount ranging from about 0.05% to about 5% by weight, relative to the weight of the hair color base composition.

20. (canceled)

21. The method according to claim 16, wherein the at least one carboxylic acid is chosen from oxalic acid, malonic acid, glutaric acid, succinic acid, adipic acid, glycolic acid, citric acid, tartaric acid, malic acid, sebacic acid, maleic acid, fumaric acid, benzoic acid, citraconic acid, aconitic acid, propane-1,2,3-tricarboxylic acid, trimesic acid, or combinations thereof.

22. (canceled)

23. The method according to claim 16, wherein the at least one carboxylic acid is present in the hair color base composition in an amount ranging from about 0.01% to about 3% by weight, relative to the weight of the hair color base composition.

24. (canceled)

25. (canceled)

26. The method according to claim 16, wherein the at least one fatty compound is chosen from C12-C22 fatty alcohols.

27. The method according to claim 16, wherein the at least one oxidation base is present in the hair color base composition in an amount ranging from about 0.001% to about 20% by weight, relative to the total weight of the hair color base composition.

28. The method according to claim 16, wherein the at least one coupler is present in the hair color base composition in an amount ranging from about 0.001% to about 20% by weight, relative to the total weight of the hair color base composition.

29-64. (canceled)
